(19) United States
(12) Patent Application Publication
(10) Pub. No.: US 2026/0196320 A1
(43) Pub. Date: Jul. 9, 2026

(54) DIGITAL THERAPEUTIC METHOD AND SYSTEM TO MONITOR AND ASSIST IN SELF-TREATING CHRONIC COUGH

(71) Applicant: HYFE Inc., Wilmington, DE (US)

(72) Inventors: Reid Moorsmith, Vancouver (CA); Paul Simon Rieger, Vienna (AT); Joseph Russell Brew, Gainesville, FL (US); Laurie Jean Slovarp, Missoula, MT (US); Jane Salois, Missoula, MT (US); Yuna Okada, Vancouver (CA); Olena Metellia, Krakow (PL); Jaume Cabecerans Bertran, Sant Cugat del Valles (ES); Mai Yamamoto, Vancouver (CA)

(21) Appl. No.: 19/367,591

(22) Filed: Oct. 23, 2025

Related U.S. Application Data
(63) Continuation-in-part of application No. PCT/US25/52187, filed on Oct. 23, 2025.
(60) Provisional application No. 63/713,451, filed on Oct. 29, 2024.
(60) Provisional application No. 63/753,501, filed on Feb. 4, 2025.

(51) Int. Cl. G16H 20/00

(52) U.S. Cl. CPC G16H 20/00

(57) ABSTRACT

A digital therapeutic method for delivering personalized Behavioral Cough Suppression Therapy through an electronic device, such as a smartphone, operates by collecting both objective data (such as cough sounds detected by the device's microphone and analyzed by a processor) and subjective data (self-reported by the patient through the app). By combining these two data streams, the device automatically generates personalized recommendations for cough suppression strategies. The method includes detecting patterns in cough events, such as trends over time, bouts of coughing, or periods of reduced coughing, and adjusting recommendations dynamically. The system also tracks adherence to therapy, guides patients through suppression training sessions, and generates progress reports. It can elicit potential cough triggers and deliver specific advice for managing them. Importantly, the recommendation engine can use machine learning to refine its suggestions based on ongoing data, enabling increasingly tailored and effective support.

### CROSS-REFERENCE DATA

This patent application is a continuation-in-part and claims a priority date benefit from the PCT Application No. PCT/US 25/52187 with the same title and filed on Oct. 23, 2025. This application also claims a priority benefit from the following two U.S. Provisional Patent Applications: No. 63/713,451 filed on Oct. 29, 2024, and entitled “Digital therapeutic to treat chronic cough,” and No. 63/753,501 filed on Feb. 4, 2025, and entitled “Digital therapeutic method to monitor and assist in self-treating chronic cough,” all of which are incorporated herein by reference in their respective entireties.

### BACKGROUND

Without limiting the scope of the invention, its background is described in connection with methods, systems, and therapies for treating chronic cough. More particularly, the invention describes a method and a system that can be implemented as a smartphone application to automatically monitor the patterns of chronic cough and develop recommendations for applying a therapy aimed at reducing the symptoms of chronic cough.

Chronic cough, defined as a cough lasting more than 8 weeks, is a prevalent condition worldwide, affecting approximately 5-10% of the adult population. It is more frequently observed in women and older individuals aged 50-70 years. Chronic cough can arise as a primary disorder or as a symptom of underlying diseases, such as asthma, gastroesophageal reflux disease (GERD), upper airway cough syndrome (UACS), or chronic obstructive pulmonary disease (COPD). Risk factors include smoking, environmental pollution, occupational irritants, and certain medications (e.g., angiotensin-converting enzyme inhibitors).

Diagnosis of chronic cough requires a thorough history and clinical examination to determine its etiology. Physicians typically evaluate key characteristics such as the cough's onset, duration, triggers, and associated symptoms (e.g., wheezing, nasal congestion, or heartburn). Initial assessments may include a chest X-ray to rule out serious respiratory pathologies, such as malignancy or pulmonary fibrosis. Additional investigations may involve pulmonary function testing, bronchoprovocation testing (for asthma), sinus imaging (for UACS), and pH monitoring to assess for GERD-related cough. The presence of red-flag symptoms, such as hemoptysis, significant weight loss, or persistent dyspnea, requires further evaluation to exclude malignancy or systemic diseases.

The management of chronic cough involves identifying and treating the underlying cause. In cases where asthma or asthma-like conditions are diagnosed, inhaled corticosteroids and bronchodilators are prescribed. For certain coughs, a combination of antihistamines, nasal corticosteroids, and decongestants may be employed. GERD-related cough is managed through lifestyle modifications (e.g., dietary changes, weight loss) and proton pump inhibitors. Smoking cessation and avoidance of environmental irritants are crucial components of treatment for chronic cough due to COPD or other respiratory diseases. In unexplained or refractory chronic cough (UCC/RCC), where no underlying cause is identified or the original cause has resolved, neuromodulation therapies such as gabapentin, pregabalin, or speech therapy techniques may be utilized. Additionally, cough suppressants like codeine or dextromethorphan may provide symptomatic relief, though their use is generally limited due to side effects and the potential for dependency.

Despite prior efforts, there is no single comprehensive tool allowing for the detection and management of this condition in a satisfactory manner.

A growing body of evidence supports the efficacy of Behavioral Cough Suppression Therapy (BCST) and related interventions for patients with refractory or unexplained chronic cough. Behavioral Cough Suppression Therapy is a structured, non-pharmacological intervention that trains patients with chronic refractory or unexplained cough to recognize the sensations that precede coughing and apply targeted strategies to suppress or reduce the cough response.

The primary objective of BCST is to empower patients to voluntarily suppress their cough and reduce the heightened sensitivity of their cough reflex. This is achieved through the use of targeted strategies, including controlled breathing techniques, swallowing exercises, and mindfulness practices, which collectively help patients manage the sensation of an urge to cough—often described as a “tickle” or “itch” in the throat. These techniques are designed to interrupt the reflexive coughing cycle and provide patients with tools to manage their symptoms independently, enhancing their autonomy over the condition.

The therapy leverages the principles of neuroplasticity to recalibrate the nervous system and reduce its sensitivity to cough triggers. This involves creating new neural pathways through a process of behavioral modification. Specifically, patients learn to suppress their cough when they feel the urge, which interrupts the hypersensitive coughing response. Over time, as the brain is repeatedly “taught” that coughing is not necessary in response to these sensations, the frequency and intensity of the urge to cough diminish. This iterative cycle of suppression and desensitization facilitates a long-term reduction in cough reflex sensitivity and symptom burden.

Delivered traditionally by speech-language pathologists or physiotherapists, and increasingly through digital or telehealth platforms, BCST emphasizes patient education, skill acquisition, and consistent practice, making it a safe, effective, and evidence-based approach to improving cough-related quality of life.

Randomized controlled trials, meta-analyses, and retrospective studies consistently demonstrate that Behavioral Cough Suppression Therapy leads to significant improvements in cough frequency, severity, and cough-related quality of life. For example, Vertigan et al. (2006) and Chamberlain Mitchell et al. (2017) both conducted randomized controlled trials showing substantial symptom reduction and improved health-related quality of life following speech pathology or physiotherapy-based cough suppression interventions. More recent evidence, including Yi et al.'s (2024) meta-analysis, confirms these therapeutic benefits across multiple trials, highlighting BCST as a robust, evidence-based approach for chronic cough management. Similarly, Patel et al. (2011) and Ryan et al. (2010) reported significant gains in cough-specific health outcomes and cough reflex sensitivity, further validating the effectiveness of structured behavioral therapy protocols.

Published evidence also shows that Behavioral Cough Suppression Therapy is adaptable to modern delivery methods, increasing its accessibility. Salois et al. (2025) demonstrated that internet-based Behavioral Cough Suppression Therapy (iBCST) yields outcomes comparable to traditional in-person therapy, while Sundholm et al. (2022) found positive results when Behavioral Cough Suppression Therapy was delivered via telehealth. These findings suggest that digital and remote modalities can preserve the therapeutic benefits of Behavioral Cough Suppression Therapy while reducing barriers to care. Furthermore, Wright et al. (2021) reported that treatment benefits persist long term, with many patients experiencing durable improvement or elimination of cough beyond six months. Slovarp et al. (2021) also emphasized that early intervention with Behavioral Cough Suppression Therapy is both clinically effective and potentially cost-efficient. Collectively, these studies provide strong and consistent evidence that behavioral cough suppression therapy is not only effective but also versatile in delivery, making it a promising frontline intervention for patients with chronic refractory cough.

However, the availability of this therapy is limited by the fact that it is only administered by about 200 speech-language pathologists in the United States, and far fewer in other countries.

The need exists, therefore, for a method and system to monitor cough, disseminate and personalize/individualize Behavioral Cough Suppression Therapy recommendations, develop purpose-specific BCST steps, and automatically assist the patient in their implementation and progress monitoring.

### SUMMARY

Accordingly, it is an object of the present invention to overcome these and other drawbacks of the prior art by providing a novel approach to develop personalized, purpose-specific recommendations for applying Behavioral Cough Suppression Therapy and monitoring its results.

It is another object of the present invention to provide a novel method for automatically generating and providing personalized recommendations for applying Behavioral Cough Suppression Therapy based on objective and subjective cough data.

It is a further object of the present invention to provide a digital application of the method for use with a smartphone or another electronic device.

Described herein is a novel, comprehensive digital method for automatically generating and delivering personalized Behavioral Cough Suppression Therapy using an electronic device, such as a smartphone, smartwatch, or smart ring. The system integrates both objective cough tracking data, such as sounds captured via the device's microphone and processed internally or externally to detect cough events, and subjective patient-reported data, which may be entered through a user interface on the same device. By analyzing these two inputs together, the system produces individualized recommendations for applying Behavioral Cough Suppression Therapy in real time.

The novel method extends beyond simple monitoring to include adaptive personalization. The system may detect trends in coughing behavior, such as increasing or decreasing frequencies, the occurrence of cough bouts, or periods without coughing, and dynamically adjust therapy recommendations in response. This adaptability also accounts for subjective patient-reported outcomes, adherence to therapy exercises, and historical engagement with the intervention. To reinforce engagement, the system can deliver tailored encouragement when cough events decline or provide supportive messages when cough persists despite good adherence.

Additional features may include guiding patients through structured Behavioral Cough Suppression Therapy training sessions directly on the device, tracking adherence to these sessions, and providing periodic progress reports. The system may also identify individual cough triggers, provide educational materials on them to the patient, and generate specific coaching on how to manage or overcome those triggers. Recommendations and feedback can be delivered via display, speaker, or haptic cues (such as vibrations) to remind users to practice suppression strategies at the appropriate time.

At the core of the system is a Behavioral Cough Suppression Therapy recommendation engine, which may employ machine learning algorithms to continuously refine its guidance. This engine allows the therapy to improve over time, becoming more effective as it learns from each patient's data and broader population patterns.

In certain embodiments, a novel system is provided that includes an electronic device having a microphone configured to detect ambient sound and generate audio data corresponding to cough events; at least one processor configured to analyze the audio data to determine a history of cough events; a user interface configured to collect subjective patient-reported cough data; and a Behavioral Cough Suppression Therapy (BCST) engine configured to analyze both the history of cough events and the subjective patient-reported cough data to automatically generate and provide personalized recommendations for applying BCST. In some embodiments, the electronic device is selected from a smartphone, a smartwatch, or a smart ring, and the BCST engine may be implemented either on the device or on an external processor in communication with the device via a network connection. The user interface may be further configured to collect subjective patient-reported outcomes using at least one of a numerical rating scale, a visual analog scale, or a patient global impression of severity scale.

The BCST engine may be configured to detect a trend in cough events and to adjust the therapy recommendations based thereon. In certain implementations, the BCST engine further detects a trend in subjective patient-reported outcomes, records a history of adherence to therapy, and adjusts the recommendations based on both trends and adherence. The engine may identify cough trends, cough bouts, and gaps with no coughing, and may generate personalized recommendations through visual, auditory, or haptic signals using a display, speaker, or vibration generator of the device. Positive reinforcement messages may be provided when a subsiding trend of cough events is detected, while encouraging support messages and recommendations to review appropriate elements/modules of the therapy to improve results may be generated upon detecting an increasing trend of cough events.

In some embodiments, the BCST engine generates periodic progress reports summarizing changes in cough frequency, cough bouts, and subjective patient-reported outcomes. The engine may identify at least one cough trigger and provide patient specific guidance for navigating the identified trigger. As used herein, a cough trigger is any external or internal factor that precipitates or exacerbates a cough event. Cough triggers may be discovered through one or more of the following means:
Patient Self-Reporting via the user interface, for example when the patient is prompted (e.g., via guided questions or checkboxes) to indicate known triggers such as dry air, environmental allergens, certain foods, strong odors, or post-nasal drip,
Machine Detection by correlating spikes in objective cough event frequency with contextual sensor data (e.g., humidity sensors detecting dry air, accelerometer data identifying eating motions),
Guided Questionnaires that help patients systematically test and record potential triggers by performing structured exposure exercises (e.g., sipping dry versus moist foods) and noting subsequent cough responses.

In embodiments, cough triggers may include, without limitation, at least one of the following:
Dry air (e.g., low humidity environments)
Dry or coarse foods (e.g., crackers, chips)
Drinking
Cold air exposure
Strong odors or fumes (e.g., perfumes, cleaning agents)
Post-nasal drip or throat mucus
Gastroesophageal reflux episodes
Physical exertion
Talking
Singing
Laughing
Changing body position (such as standing up or lying down)
Smoke
Dust.

Once a cough trigger is identified, whether via patient self-reporting, machine-aided analysis, or guided questioning, the system may provide patient-specific guidance on managing or avoiding that trigger. For example, upon identifying dry air as a trigger, the application may recommend using a humidifier or wearing a face mask in low-humidity settings.

While initial implementations may rely on subjective patient-reported cough triggers collected through the user interface, the architecture may support future integration of automated trigger detection modules. Such modules may analyze temporal alignments between objective cough events and environmental or physiological sensor readings to propose additional triggers to the patient for confirmation or avoidance planning.

The engine may be trained and operated using a machine-learning algorithm configured to adaptively refine therapy recommendations based on ongoing user data( and may employ reinforcement learning to iteratively refine feedback strategies based on prior user engagement and clinical outcome data. The at least one processor may be configured to automatically operate the microphone for at least 10 hours per day to collect sufficient data for analysis and to collect baseline cough data prior to the initiation of BCST. The BCST engine may guide the patient through a structured cough suppression training session and monitor adherence to the training. In certain embodiments, a smartphone processor executes a mobile application implementing the BCST engine and communicates with a remote database and physician dashboard via an application programming interface (API). The dashboard may be configured to display objective cough data, subjective patient-reported outcomes, and adherence metrics for remote review by a clinician.

Overall, the novel method has the potential to establish a digital therapeutic platform that transforms Behavioral Cough Suppression Therapy from an episodic, clinician-delivered intervention into a continuous, adaptive, and data-driven therapy capable of offering real-time, personalized coaching.

### BRIEF DESCRIPTION OF THE DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 is a block diagram of the main components and their interactions during implementation of the novel method,

FIG. 2 is a first example of smartphone user interface tracking cough suppression progress,

FIG. 3 is a second example of such a user interface,

FIG. 4 is the first chart showing the results of a pilot study of implementing the method of the present invention,

FIG. 5 is a second chart illustrating the results of the same study,

FIG. 6 is the first panel of an exemplary doctor's dashboard illustrating a patient's progress in treating chronic cough using the therapy recommendations of the present method,

FIG. 7 is a second panel of the same,

FIG. 8 is a third panel of the same,

FIG. 9 is an exemplary screenshot showing the initial assessment stage of the therapy,

FIG. 10 is an exemplary screenshot showing the goal-setting stage of the therapy,

FIGS. 11 and 12 are the first and second exemplary panels of a screenshot showing Week 1 learning (cough 101) stage of the therapy,

FIGS. 13 to 15 are the first, second, and third exemplary panels of a screenshot showing Week 1 learning (Understanding Cough Sensitivity) stage of the therapy,

FIGS. 16 to 18 are the first, second, and third exemplary panels of a screenshot showing Week 2, the cough control breathing suppression technique, stage of the therapy,

FIGS. 19 and 20 are the first and second exemplary panels of a screenshot showing Week 2, readiness to suppress when the urge to cough arises, stage of the therapy,

FIGS. 21 to 23 are the first, second, and third exemplary panels of a screenshot showing Week 3, training: part 2, the chin-tuck swallow suppression technique, stage of the therapy,

FIG. 24 to 27 are the first, second, third, and fourth exemplary panels of a screenshot showing Week 3, training: part 2, stage of the therapy,

FIG. 28 is an exemplary screenshot of Week 4, technique refinement stage,

FIGS. 29 and 30 are the first and second exemplary panels of a screenshot showing Week 4, optimizing progress, stage of the therapy,

FIGS. 31 and 32 are the first and second exemplary panels of a screenshot showing Week 5, sustaining progress: navigating triggers, stage of the therapy,

FIGS. 33 to 37 are the first, second, third, fourth, and fifth exemplary panels of a screenshot showing Week 5, trigger exposure, stage of the therapy, and

FIGS. 38 to 41 are the first, second, third, and fourth exemplary panels of a screenshot showing Week 6, planning for the future, stage of the therapy.

### DETAILED DESCRIPTION OF PREFERRED EMBODIMENTS OF THE INVENTION

The following description sets forth various examples along with specific details to provide a thorough understanding of the claimed subject matter. It will be understood by those skilled in the art, however, that claimed subject matter may be practiced without one or more of the specific details disclosed herein. Further, in some circumstances, well-known methods, procedures, systems, components and/or circuits have not been described in detail in order to avoid unnecessarily obscuring claimed subject matter. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

The novel method of automatically generating and providing personalized recommendations for applying Behavioral Cough Suppression Therapy may include the following three main steps:
a. collecting objective cough tracking data by operating an electronic device having a microphone and (i) configured to communicate sound detected by the microphone to an external processor or (ii) comprising an internal processor configured to analyze sound detected by the microphone and determine a history of cough events therefrom,
b. collecting subjective patient-reported cough data by providing a user interface configured for entry of subjective patient-reported cough data, and
c. operating the external processor or the internal processor to analyze both objective cough tracking data and subjective patient-reported cough data to automatically generate and provide personalized recommendations for applying Behavioral Cough Suppression Therapy.

These steps are now described below in greater detail.

Objective cough data may be collected using various known techniques and methods. One suitable approach is described in our U.S. patent application Ser. No. 18/429,130 filed Jan. 31, 2024, now U.S. Pat. No. 12,004,851, and U.S. patent application Ser. No. 18/738,180 filed Jun. 10, 2024, which are incorporated herein in their respective entireties by reference. These patent documents describe novel methods for automatically detecting cough events using continuous audio monitoring and advanced signal analysis. The process begins with recording ambient sound and identifying possible cough events whenever a change in sound energy exceeds a set threshold. Short audio snippets surrounding each potential event are then captured and analyzed in the frequency-time domain, focusing on energy above 100 Hz, to distinguish coughs from non-cough sounds. Non-cough sounds are discarded, while verified cough events are logged, optionally with time stamps, to compile a comprehensive record of cough activity. Described are techniques to improve accuracy and reliability, such as applying additional thresholds, dividing audio into overlapping time frames, and filtering out silence. Classification of cough events can be performed using statistical models, including neural networks, with convolutional neural networks (CNNs) highlighted as a preferred method. These models may be pre-trained on large datasets of known cough and non-cough recordings to assign probability scores for accurate event detection. The method also refines event capture by allowing snippets to start slightly before the detected onset and limiting total recording length to under one second.

The present invention is not limited to these specific examples, as other known techniques and devices may also be used for the purposes of the invention.

A variety of technologies can be employed to record and process cough sounds for objective tracking and analysis. At the most basic level, this involves a microphone capable of continuously recording ambient sound in the user's environment. Such microphones may be embedded in widely available wearable consumer devices such as smartphones, smartwatches, or smart rings, or they may be integrated into specialized wearable sensors designed for health monitoring. These devices can either transmit raw audio data to an external processor, such as a local computer or a cloud-based server, or perform on-device processing using an internal processor. In the latter case, modern mobile devices are often powerful enough to execute algorithms locally, reducing the need for constant data transfer and enabling real-time cough detection.

The processor responsible for implementing cough analysis may take the form of a smartphone, tablet, or other portable device that runs dedicated software or applications. These programs can perform advanced signal processing, extracting features such as acoustic energy distribution, frequency content, and temporal patterns to differentiate coughs from non-cough sounds. In some implementations, edge computing techniques may allow the device itself to classify cough events, while other approaches rely on cloud-based machine learning models trained on large datasets of known cough and non-cough recordings. Wearables, such as smart rings or watches, add further advantages by enabling discreet, continuous, and passive monitoring, with vibration or haptic feedback capabilities to deliver real-time prompts or reminders. Any of these modern technologies, alone or in combination, may provide a versatile ecosystem for recording and analyzing cough sounds, enabling continuous, objective monitoring that can support personalized cough suppression therapy and disease management.

Step (a) of objective cough data collection may be conducted intermittently or continuously. In embodiments, step (a) may be implemented for at least 10 hours per day, so as to collect sufficient data to improve the accuracy of the method. In other embodiments, step (a) may be conducted for at least 12 hours per day, at least 16 hours per day, at least 20 hours per day, or continuously without interruption.

Similarly, step (a) may be preceded by a step of collecting baseline cough data, when the patient is not engaged in any cough suppression techniques, including self-guided techniques. In embodiments, a step of collecting baseline data may be conducted for a period of at least 3 consecutive days, at least 4 consecutive days, at least 5 consecutive days, at least 6 consecutive days, at least 7 consecutive days, or at least 8 consecutive days.

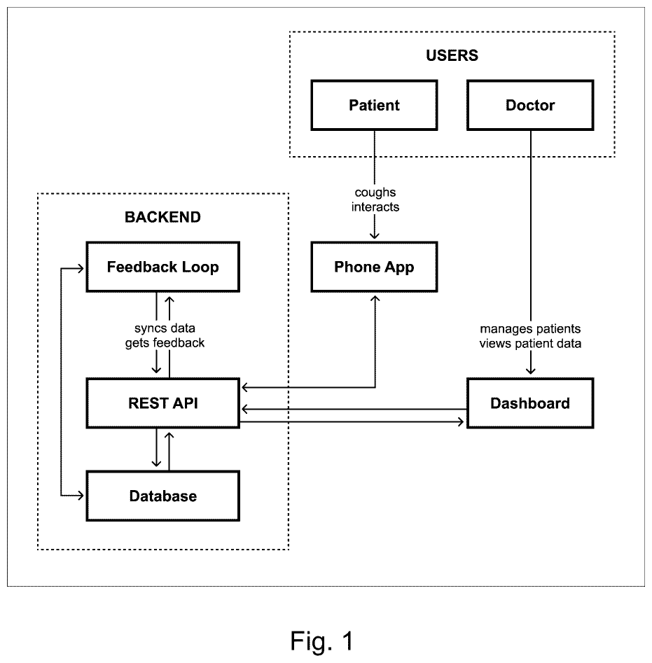

One example of the architecture configured to support the method of the invention is seen in FIG. 1. The diagram in this figure illustrates a digital therapeutic system for cough management, showing the interaction between users, frontend interfaces, and backend components.

The system may involve two main categories of users: patients and doctors. Patients interact primarily with the phone application, which serves as their main interface. Through the app, patients may engage with therapy modules, log practice sessions, and receive real-time feedback on their cough suppression efforts. The app may also be capable of detecting coughs automatically and collecting subjective patient-reported outcomes. Doctors, on the other hand, may use the dashboard, which presents aggregated patient data in a structured and interpretable format. This allows clinicians to review patient progress, adherence, and clinical outcomes without direct interaction with the patient through the app.

On the frontend, the phone application and the dashboard serve distinct but connected purposes. The phone application automatically captures cough activity and records self-reported patient interactions, syncing these data with the backend via the REST API. It may also receive feedback from the backend and deliver personalized guidance and motivational messages to the patient. The dashboard, designed for doctors, may retrieve processed data through the REST API and present it in graphical displays, adherence metrics, and progress summaries, enabling clinicians to monitor and manage patients efficiently.

The backend may consist of three interconnected components: the feedback loop, the REST API, and the database. The feedback loop functions as the decision-making engine. It processes both objective cough measures, such as cough frequency and patterns, and subjective patient-reported outcomes, such as symptom severity and adherence levels. Based on this analysis, it automatically generates targeted recommendations, including therapy instructions and motivational feedback, which may then be sent to the phone application through the REST API. The REST API may act as the communication hub that enables data exchange between the phone application, the dashboard, and the backend components. The database stores all patient-related information, including raw cough data, adherence records, progress indicators, and feedback history. These stored data are made available to the feedback loop for ongoing analysis and to the dashboard for physician review.

The interaction across the system creates an adaptive feedback loop. The patient provides objective and subjective data through the phone application, which is then transmitted to the backend. The feedback loop processes the data, generates tailored interventions, and delivers them back to the patient through the application. Simultaneously, doctors can access the dashboard to view up-to-date information, track patient adherence, and assess clinical outcomes. This closed-loop design allows the digital therapeutic to actively adjust treatment recommendations in response to the patient's behavior and progress while keeping clinicians informed for oversight and management.

Division of the functions between the front end and the back end is not the only way to practice the invention. In other embodiments, all actions may be performed by a single electronic device, or may be split between more elements, not necessarily located together, as the invention is not limited in this regard.

Additional measures of objective cough event detection in step (a) and further analysis may be included to provide a more complex, complete picture of the patient's cough patterns and may include:
Cough Trend: Using the Kruskal-Wallis statistical test, this feature shows whether a patient's cough has changed relative to recent days, weeks, or months. To measure the daily change, Cough Trend takes the hourly cough counts from the past 24 hours and compares them to the hourly cough counts from the preceding three days—the color and angle of the Cough Trend arrow are determined by the fluctuations among these counts;
Bouts are coughs that are clustered together without a cougher having time to catch their breath. This is important because coughs without a break are particularly taxing to those with a problematic cough;
Gaps with no cough show the white space in between coughs, when people with a problematic cough can literally and figuratively catch their breath. A gap may be defined as lasting 15 minutes or longer, containing zero coughs. This measure shows patients how many breaks from coughing they get in a day, week, or month, and when these breaks occur.

In addition to step (a) of collecting objective cough data, the present invention teaches a step (b) of collecting subjective, subjective patient-reported cough data. This step may be performed by providing a user interface configured for manual entry of cough-related data into the electronic device, such as the same smartphone that may be used in the first step of the process. The invention is not limited to using the same electronic device for both the first step and the second step, though, as different electronic devices may also be used to accomplish different steps of the method.

Self-reported cough data may also be entered by a caregiver, family member, or medical personnel, as the invention is not limited in this regard.

In broad terms, subjective cough data may include the frequency of attempts to suppress coughing, the perceived success of these suppression efforts, and self-assessed success rates. Patients may also report changes in the sensation of the urge to cough over time. This nuanced data can provide critical insights into the dynamics of cough suppression and the effectiveness of intervention strategies, contributing to a comprehensive understanding of the interplay between subjective symptom burden and patient management behaviors.

Subjective cough data may include reporting using known metrics of cough monitoring. In embodiments, self-reported data may be in the form of a subjective patient-reported outcome. Patient-reported outcomes (PROs) are a valuable methodological approach for collecting subjective information regarding chronic cough events, offering insights into the patient's experience and perception of their condition. Established PRO instruments, such as the cough Numerical Rating Scale, Patient Global Impression of Severity (PGI-S), and Visual Analog Scale (VAS), may be utilized to systematically evaluate and self-report the severity of the cough and its broader impact on the patient's quality of life. These already validated tools may provide standardized metrics that facilitate comparability and reliability in assessing the burden of chronic cough from the patient's perspective.

The self-reporting process of cough events may be structured around four targeted questions integrated into the digital therapeutic app, designed to capture the patient's awareness, effort, and success in applying cough suppression strategies. Patients may be prompted to reflect on their experiences by reporting on these exemplary questions:
1. how often they perceive an urge to cough before the cough occurs,
2. how frequently they attempt to suppress the cough once the urge arises,
3. how often these suppression attempts successfully prevent the cough altogether, and
4. how consistently they are able to regain control and reduce the severity of coughing when suppression is not fully effective.

Together, these exemplary questions may provide a multidimensional view of both symptom perception and behavioral response. Other questions may be added to the self-reporting part of the user interface.

The self-reported responses may then be used to tailor personalized therapy coaching, including changes in real time. For instance, patients who report infrequent attempts at suppression may be reminded that consistent practice is essential for long-term success. Those who struggle with effectively preventing cough may be directed to therapy refinement materials that focus on improving technique execution. If patients indicate difficulty in reducing severity during breakthrough coughing episodes, they may be provided with targeted coaching on mid-bout control strategies. This structured feedback loop ensures that patients receive guidance aligned with their specific challenges, reinforcing the behavioral therapy principles while optimizing the therapeutic impact through personalized, adaptive intervention.

Step (c) of the method, namely operating the external processor or the internal processor to analyze both objective cough tracking data and subjective patient-reported cough data to develop a personalized Behavioral Cough Suppression Therapy, may be implemented using an adaptive feedback loop and the computer-implemented Behavioral Cough Suppression Therapy Engine.

An adaptive feedback loop may be implemented as an automated mechanism to integrate objective data on cough rates and patterns with subjective patient-reported outcomes. Data collected from the therapeutic application may be periodically transmitted to a backend system for processing, including usage statistics (e.g., time spent per training section, progress within sections, and periods of active monitoring), physiological measures (e.g., cough frequency normalized over time), and self-reported measures from in-app questionnaires. The backend aggregates this data into a feature set representing patient engagement, adherence, and clinical response to therapy.

A machine learning model may analyze this feature set to select feedback messages or instructions from a predetermined library. These messages, covering BCST guidance and psychosocial encouraging support, may be designed to reinforce adherence, clarify misunderstood material, and encourage positive therapeutic behaviors. By applying the trained model to current data, the system may dynamically tailor interventions to each patient's observed usage patterns and clinical progress.

The model may be trained on historical patient data in a supervised or semi-supervised manner, correlating feature sets with expert feedback assignments or outcomes such as cough reduction, adherence, or subjective patient-reported improvement. Reinforcement learning may also be applied, where different feedback strategies are evaluated against subsequent engagement or clinical outcomes. Training can use diverse algorithms, including decision trees, ensemble methods, regression, or neural networks, to generalize across patient behaviors and responses.

The feedback loop is aimed at optimizing Behavioral Cough Suppression Therapy by adjusting recommendations in real time. Patients log suppression practice sessions via the application, providing measurable indicators of adherence. This data allows the system to assess effort and deliver personalized messages that reinforce engagement and encourage progress. The loop also automatically detects changes in objective cough measures, such as frequency and clustering of bouts, and adapts therapy steps accordingly.

When objective improvements are detected, such as a reduction in cough frequency, for example, the computer system will automatically generate and deliver positive reinforcement, such as rewards, to enhance patients'sense of accomplishment and motivation. Sample positive reinforcement and encouragement messages may include the following: “Great job practicing your techniques! Your commitment will pay off,” or “I know it's not easy when you start practicing. Don't worry, you'll see the dividends when you are facing one of your cough triggers in daily life,” or “Almost there! Let's do 3 more Cough-Control Breathing reps, and you get a star for this session!”

Conversely, if cough rates remain static, the processor may be configured to automatically generate and provide targeted coaching to refine cough suppression techniques and offer practical strategies for effective implementation in daily life. In cases where cough measurements worsen, the system will automatically focus on addressing adherence and reinforcing proper techniques through supportive coaching.

By continuously integrating data from both objective cough measurements and subjective cough data reported by the patient, the feedback loop ensures that the recommended therapy strategy remains responsive to patients'efforts and evolving needs. It maintains a comprehensive record of cough suppression attempts, tracks changes in cough patterns, and adapts guidance based on the patient's perceived and measured success. This iterative, patient-centered automatic approach enhances the efficacy of the cough suppression therapy by aligning therapeutic interventions with real-time patient progress, fostering greater adherence, and optimizing outcomes.

Automated generation and delivery to the patient of personalized therapy recommendations may be done using a computer-implemented Behavioral Cough Suppression Therapy Engine capable of adapting to complex, purpose-specific patterns of patient behavior and response. The core objective is to maximize the effectiveness of therapy delivery by tailoring the sequence of interventions, such as those recommended via videos, text messages, or notifications, to specific user profiles and circumstances.

The Behavioral Cough Suppression Therapy Engine may be used first to define a measurable “effectiveness” metric to evaluate intervention success. This could include behavioral engagement (e.g., number of screen views or time spent interacting with content), clinical outcomes (e.g., changes in cough frequency or intensity), or user-reported feedback (e.g., sleep disturbance or urinary incontinence). Input variables, or “features,” might encompass demographic information (e.g., age, gender), device characteristics (e.g., operating system), temporal factors (e.g., time of day), and dynamic health indicators (e.g., recent changes in cough rates, heart rate, heart rate variation, respiratory rate, or other health indicators). By analyzing these inputs, the model can identify patterns that correlate with successful therapy outcomes.

The computer-implemented Engine may operate as an automated, dynamic, data-driven therapy steps-generating system, employing an iterative learning process to optimize content delivery over time. Initially, the algorithm of the Engine may predict the most effective intervention for each user based on historical data and current circumstances. For instance, it may analyze whether a user tends to engage with video content longer at specific times of the day or respond positively to motivational messages under certain conditions.

The computer-implemented Engine may learn by continuously updating its predictions as new data becomes available. For example, if early interventions are effective in reducing cough frequency by 40%, the Engine can iteratively refine its approach to incorporate new data and feedback. This may potentially achieve greater efficacy, such as improving to 55% cough frequency reduction.

The Behavioral Cough Suppression Therapy Engine may decide on the best approach to generating therapy recommendations based on available data. Given the constraints of data availability, the Engine may elect to use simpler probabilistic models like Hidden Markov Models (HMMs), which might be more practical than advanced neural network architectures (e.g., RNNs, Transformers). HMMs can model sequential data and learn the probability of transitions between states, making them suitable for predicting the impact of different interventions on evolving user conditions.

The BCST Engine may also be configured to automatically leverage fine-grained data to make predictions that would not be feasible for human intuition. It evaluates hundreds of variables, such as whether a user opens a specific type of notification, the duration spent viewing content, or recent trends in cough patterns. For instance, the model might predict that a user who spends less time on a particular lesson would benefit more from shorter, targeted reminders, whereas another user might respond better to comprehensive instructional videos.

The Engine may also be configured to account for individual variability in intervention outcomes. For example, two users receiving the same recommendations video might exhibit different responses depending on their demographic profile, device type, and health trajectory. By incorporating such predictors into its decision-making process, the Engine can automatically identify optimized content and timing for each individual, thereby enhancing therapy effectiveness and minimizing user fatigue.

The personalization of therapy recommendations results from the analysis of the messages and coaching the patient receives compared to their progress in being able to suppress their cough. The Engine may combine objectively recorded cough rate and subjective patient-reported data over the first two weeks of the treatment to provide suggestions, guidance, and encouragement that produce optimal results. Over time, the Engine may be configured to continue to learn which patterns respond to which suggestions, guidance, and encouragement, and uncover latent predictors of therapy success and integrate these insights to deliver increasingly precise and effective therapy steps. This would allow the Engine to provide more personalized, optimized recommendations to the particular patient.

The Engine may be further configured to assist the patient via the smartphone app in identifying cough triggers. In the Navigating Triggers portion of the user interface, the app may guide patients in identifying which common situations and stimuli provoke their cough. Most chronic cough patients are familiar with their own triggers when they begin training. For those who aren't, they are guided through a series of questions to help those who aren't aware to be able to apply the overall approach, which can be described as “Be Ready, Respond to the Urge (to cough), and Regain Control.”

### INTEGRATION AND ANALYSIS OF OBJECTIVE AND SUBJECTIVE COUGH DATA

In embodiments of the invention, the processor may operate a multi-stage data fusion and analysis pipeline to integrate objective cough tracking data and subjective patient-reported cough data into unified, personalized Behavioral Cough Suppression Therapy recommendations. The following describes this multi-stage process:

### Preprocessing of Objective Cough Tracking Data

a. Raw audio data captured by the device microphone undergoes noise reduction and band-pass filtering to isolate cough-like acoustic signatures.
b. Signal-processing algorithms detect discrete cough events, timestamp each event, and extract features such as cough duration, inter-cough interval, frequency, amplitude, and temporal patterns (e.g., cough bouts vs. isolated coughs).
c. Processed cough features are stored in an objective cough event history database.

### Preprocessing of Subjective Patient-Reported Cough Data

a. Subjective patient-reported data (e.g., numerical ratings of cough severity, visual analog scale values, and free-text symptom notes) entered via the user interface are validated and normalized to a common scale.
b. Time stamps from patient entries are aligned to the same time base as the objective cough events.
c. Structured data sets are created for each reporting interval (e.g., hourly or daily buckets), including aggregated patient severity scores and annotations.

### Data Alignment and Synchronization

a. The processor correlates each subjective patient-reported entry with the nearest objective cough event cluster based on time stamps.
b. A joint time series data structure is generated, containing paired samples of objective metrics (e.g., cough count per hour) and subjective metrics (e.g., reported severity per hour).
c. Where reporting intervals lack direct objective events, interpolation or imputation algorithms estimate objective metrics; conversely, low-adherence reporting intervals are flagged.

### Feature Extraction and Trend Analysis

a. The fused time series is analyzed to extract composite features such as cough-severity co-trends, divergence between objective and subjective measures, adherence correlation, and latent patterns (e.g., patient over-or under-reporting relative to objective cough burden).
b. Statistical methods (e.g., moving averages, exponential smoothing) detect trends and change points in both data streams, informing whether cough frequency is increasing, decreasing, or plateauing relative to patient perception.

### Personalized Recommendation Generation

a. The BCST engine applies decision logic or a trained machine-learning model that takes fused features as input and outputs therapy adjustments. Input features include recent cough frequency, patient severity ratings, trend direction, bout patterns, and adherence history.
b. Rules or model outputs determine recommended exercises (e.g., breathing training, swallow-technique reminders), timing (e.g., suppression cue after a bout), intensity (e.g., length of training session), and feedback modality (visual, auditory, or haptic prompts).
c. Reinforcement learning components update model parameters over time, using outcomes (both objective reduction and subjective relief) to refine recommendation strategies.

### Continuous Feedback Loop

a. Following delivery of a recommendation, subsequent objective and subjective data are monitored to assess efficacy.
b. The processor computes a response score combining objective cough reduction and patient-reported improvement.
c. This score serves as a reward signal to the learning algorithm, closing the loop for adaptive personalization.

By implementing the above integration and analysis pipeline, the processor may be configured to seamlessly combine real-time cough acoustics with user-reported symptomology to derive highly tailored BCST recommendations, ensuring that therapy guidance dynamically reflects both measurable cough burden and patient experience.

The digital therapeutic platform of the invention may be designed to adapt to different patient scenarios. When cough frequency remains elevated while treatment engagement is low and patient-reported outcomes remain stable, the Behavioral Cough Suppression Therapy Engine may interpret this as a signal that the patient is not yet fully invested in applying therapy techniques despite their quality of life being unaffected. In this case, the Engine may emphasize motivational support, helping the patient reframe the importance of active participation and guiding her to prepare for personal triggers she has previously identified. This “high-touch” approach ensures that the patient feels supported and encouraged to reinvest in the therapy process.

In situations where cough frequency remains elevated but the patient is actively engaging with the therapy, and self-reported outcomes reveal worsening quality of life, the Behavioral Cough Suppression Therapy Engine may shift its focus toward psychosocial encouragement. Here, patients are reminded that setbacks are part of the therapeutic process and are offered structured support for managing frustration, anxiety, or discouragement. The Engine may also provide refresher lessons on core cough suppression techniques, while emphasizing the importance of preparing in advance for environments or triggers that might provoke coughing episodes.

For patients whose cough frequency remains stable despite high engagement and neutral patient-reported outcomes, the Behavioral Cough Suppression Therapy Engine may acknowledge the stability as a positive sign while also encouraging progress. In these cases, the Engine may motivate patients to explore additional suppression strategies, refine their techniques, and set new, achievable goals, thereby avoiding therapeutic stagnation and fostering continued improvement.

If cough frequency remains unchanged but the patient shows low engagement and negative self-reported outcomes, the Behavioral Cough Suppression Therapy Engine may identify a high-risk scenario of disengagement and diminished well-being. To counteract this, the Engine may provide encouragement, break therapy into short-term, manageable goals, and offer frequent check-ins. For example, patients may be prompted to commit to practicing suppression techniques consistently over a three-day period, providing an attainable milestone that can build momentum and confidence.

Finally, when cough frequency decreases and patient engagement is high, but self-reported outcomes remain neutral, the Behavioral Cough Suppression Therapy Engine may highlight the objective improvement as evidence of real progress. Patients may be encouraged to recognize their achievements, which reinforces adherence, and may be guided toward more ambitious goals such as completing the therapy program or engaging in cough trigger challenges. In this way, the app helps patients transition from managing cough symptoms to eliminating unnecessary urges altogether, ultimately enhancing both symptom control and overall quality of life.

One of the advantages of the methods and systems of the invention is the ability to use artificial intelligence and machine learning features of the Behavioral Cough Suppression Therapy Engine to dynamically change the treatment recommendations provided to suit each patient's situation based on trends in objective cough events, trends in patient-reported cough outcomes, and history of adherence to recommendations.

The method of the present invention may be implemented in a smartphone app, which may be configured to operate the smartphone microphone to collect objective cough data, as well as provide the patient with a user interface configured to collect patient-reported cough data, as well as provide personalized therapy recommendations. The following is an example of various sections of the app designed to implement successive steps of the method of the present invention.

### ONBOARDING

The onboarding phase serves as a critical foundation for the patient's therapeutic journey, ensuring that they not only understand how to use the application but also feel confident and motivated to engage with it consistently. At the outset, patients may be welcomed with a clear explanation of the therapy's purpose, including the rationale behind Behavioral Cough Suppression Therapy, the expected benefits, and how digital delivery can provide real-time support that extends far beyond traditional once-a-week visits. This helps establish trust in the system and sets realistic expectations about the trajectory of symptom improvement.

Patients may then be introduced to the app's interface through an intuitive, step-by-step walkthrough. They learn how to navigate the dashboard, access therapy modules, track their progress, and review feedback. A key component of onboarding is setting up cough monitoring, which may involve configuring the microphone on a smartphone, smartwatch, or other wearable device. The app may provide clear instructions and interactive prompts to guide patients through permissions, calibration, and test recordings so they can see in real time how cough events are detected and logged.

To reinforce engagement, the onboarding phase may also include personalized goal-setting. Patients may be asked to reflect on their current symptoms, quality of life, and desired outcomes, which are translated into measurable objectives within the app. They are shown how their cough data, such as cough frequency trends, therapy engagement, and patient-reported outcomes, will be tracked and integrated into tailored feedback.

Finally, patients may receive a preview of what to expect in upcoming modules, including self-reporting exercises, suppression techniques, and personalized coaching. The emphasis may be on creating a sense of partnership between the patient and the digital therapeutic: the app is positioned not as a passive tool, but as an active guide that responds to their individual progress and challenges. By the end of onboarding, patients are not only operationally ready but also emotionally and cognitively prepared to engage with the therapy in a structured, consistent, and goal-oriented way.

### LEARNING

The next step in the process is the learning phase, where patients may be equipped with the essential knowledge needed to understand their condition and prepare for effective cough suppression. This stage begins with a simple introduction to the basics of coughing—how it functions as a reflex, why it occurs, and what typically triggers it. Patients may then learn about Cough Hypersensitivity Syndrome (CHS), gaining insight into how increased sensitivity can heighten the urge to cough and perpetuate the problem.

Building on this foundation, the program may explore the concepts of cough triggers and the sensation of the urge to cough, highlighting their importance in recognizing patterns and applying suppression techniques consistently. Patients may learn more about the principles of Behavioral Cough Suppression Therapy, with explanations of how cognitive and behavioral methods can retrain the brain and reduce cough reflex sensitivity.

To deepen self-awareness, patients are encouraged to “explore their cough” by observing their own patterns, tracking urge-to-cough sensations, and recording experiences in an in-app diary. This awareness sets the stage for the practical application of suppression strategies. Finally, the learning phase transitions into an introduction to cough suppression itself, motivating patients to use their new understanding to break the cycle of hypersensitivity. Short knowledge check, in the form of pop quizzes or interactive exercise, help reinforce key concepts and ensure patients are ready to move forward to the next stage of therapy.

### TRAINING

The training phase builds on the knowledge gained in the earlier steps by equipping patients with practical tools to manage and suppress their cough. Here, patients are introduced to evidence-based cough suppression strategies, such as specialized breathing and swallowing techniques, broken down into simple, step-by-step instructions to make them easy to learn and practice.

At the center of this phase is the “3 Rs” framework: Be Ready, Respond to the Urge, Regain Control. This structured approach helps patients anticipate cough triggers, apply the right techniques when the urge arises, and quickly restore control if a cough slips through.

To reinforce learning, patients engage in guided practice sessions, completing repetitions (“Reps”) and following tailored recommendations (“Recs”). These exercises not only help internalize the skills but also allow the digital therapy system to monitor adherence and provide feedback.

The program also offers practical tips for navigating difficult situations, such as environments where suppression is harder or when timing and social context create challenges. Finally, a knowledge check may be offered to ensure patients are confident in their abilities and ready to advance to more complex stages of therapy.

The method may also include a step of generating a progress report on a periodic basis, such as each week in one example. A progress report may be provided to a patient to show how their subjective self-reported outcomes are changing over time, and how their cough rate has changed by day and from week to week. In addition, patients may be informed about how the number of cough bouts is changing from day to day and from week to week.

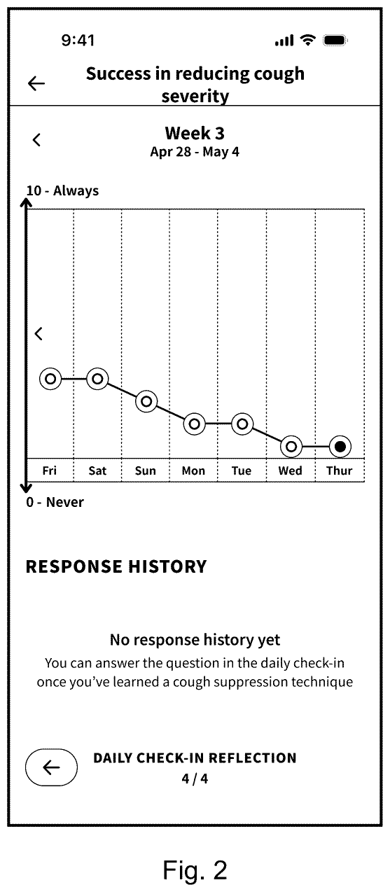

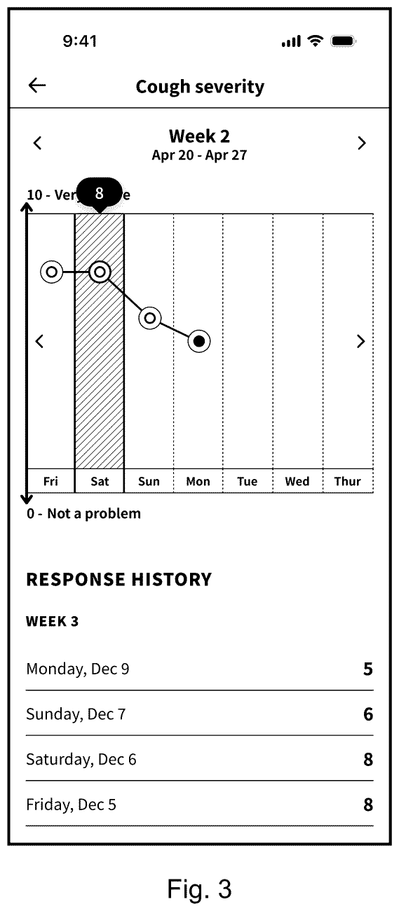

Examples of user interface showing the progress of the therapy are presented in FIGS. 2 and 3.

### OPTIMIZING AND SUSTAINING PROGRESS

The next phase of therapy focuses on adapting treatment in real time using both continuous cough monitoring data and patient-reported outcomes. Patients undergo regular progress assessments that track cough frequency, urge intensity, and adherence to practice sessions. Results are displayed in a clear, user-friendly format that highlights progress while pointing out areas for improvement. Each cough suppression training session can be guided by the device, ensuring consistency and accurate tracking.

As part of this phase, the system provides personalized refinements, adjusting recommendations to strengthen weaker areas of technique and helping patients fine-tune their skills. Patients may also be guided in navigating difficult triggers, learning to recognize specific environments or situations that intensify the urge to cough, and applying strategies to manage these challenges effectively.

Additional tailored advice may be provided to prepare patients for unique circumstances such as social gatherings, stress, or physical activity, so they feel confident applying cough suppression in real-world settings. For those with persistent triggers, the program may introduce trigger challenges, where patients gradually expose themselves to these triggers and practice suppression, building resilience and reducing sensitivity over time, an approach best suited for those who have developed solid foundational skills. Finally, the phase reinforces the importance of vocal hygiene, including hydration and rest, to support long-term respiratory and vocal health.

### PLANNING FOR THE FUTURE: COUGH FREEDOM

The final phase of therapy serves as a celebration of progress and a reinforcement of the patient's achievements in reducing coughing urges. It is designed to equip patients with the confidence and tools they need to sustain long-term success.

In this stage, patients are guided through “facing the future,” which includes learning strategies to handle any uncertainty about whether their cough might return and how to respond effectively if it does. A Future Expectations Q&A may be provided to address common questions that arise at the conclusion of Behavioral Cough Suppression Therapy, offering reassurance, motivation, and practical advice to build confidence in maintaining control.

The phase may also include a Cough Freedom Assessment, a comprehensive evaluation of the patient's current cough frequency, intensity, and overall ability to independently manage Cough Hypersensitivity Syndrome (CHS). Finally, patients may receive a progress report summarizing their journey. This report may highlight achievements, personal growth, and their individual milestones, while also providing guidance on how to preserve and strengthen their cough management skills into the future.

### PHYSICIAN DASHBOARD

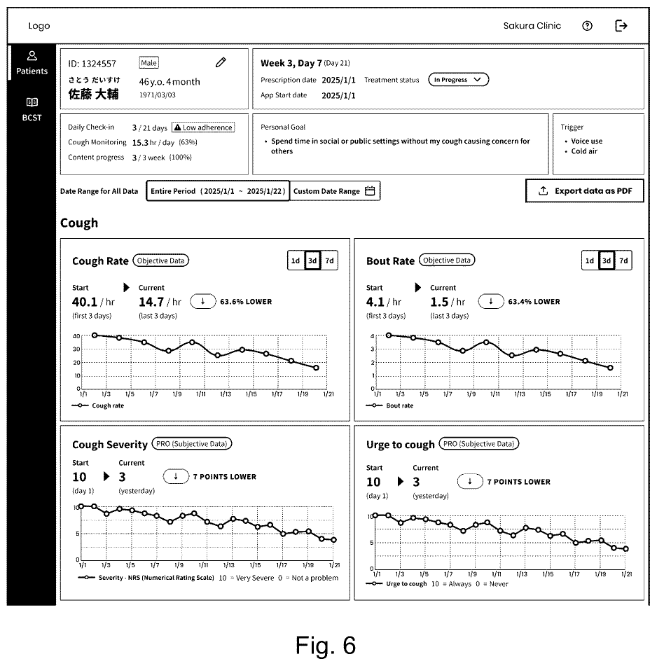

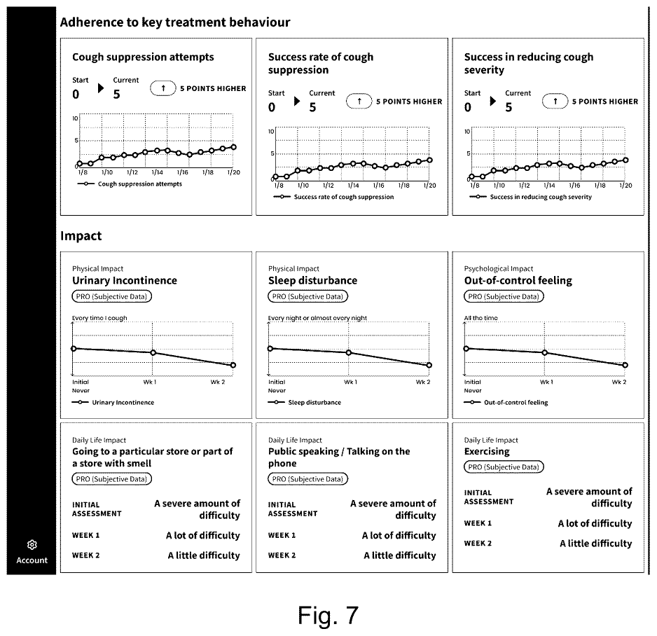

A physician-facing dashboard may also be provided as part of the overall method of the invention. Different panels of an exemplary dashboard are seen in FIGS. 6-8. The dashboard may provide a comprehensive view of a patient's progress with Behavioral Cough Suppression Therapy, combining objective cough data, patient-reported outcomes (PROs), and adherence metrics into a single interface. At the top panel (FIG. 6), it may summarize key patient details including demographics, treatment timeline, personal goals, and identified triggers. It also displays engagement indicators (FIG. 7), such as daily check-ins, cough monitoring hours, and progress through weekly modules, giving physicians a quick overview of both adherence and motivation.

Central to the dashboard are detailed cough metrics. These include objective measures such as cough rate and bout rate, which are shown as trendlines over time and paired with percent improvements compared to baseline, as well as subjective PRO measures like cough severity and the urge to cough. This integration of objective tracking with patient perceptions allows clinicians to assess both measurable and lived outcomes. The dashboard also tracks adherence to suppression techniques, highlighting the frequency of suppression attempts, the success rate in preventing cough, and the success in reducing cough severity when suppression does not fully prevent an episode.

In addition to therapeutic behaviors, app adherence is closely monitored. Metrics may include average hours of cough monitoring per day, the number of daily check-ins completed, and time spent on the app, all of which are visualized to reveal patterns of engagement. Weekly progress is tracked through completion of therapy content and scheduled check-ins, enabling clinicians to see how consistently patients are advancing through the program.

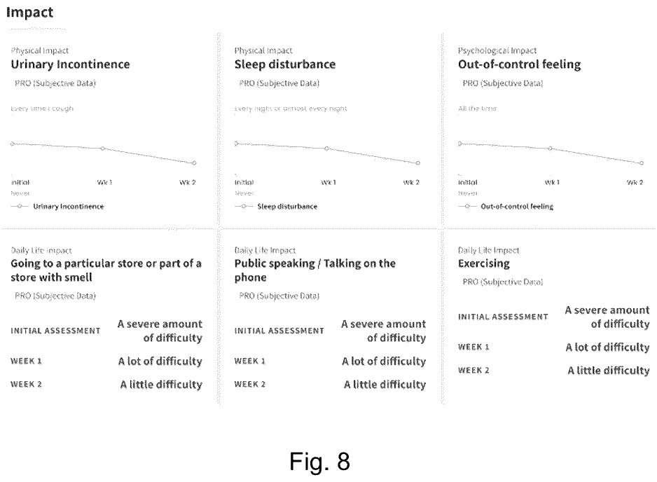

Finally, the dashboard may highlight the impact of cough on daily life (FIG. 8). This may include physical impacts such as urinary incontinence and sleep disturbance, psychological impacts such as the sense of being “out of control,” and functional challenges in areas like exercising, public speaking, or shopping. These may be tracked from initial assessments through weekly intervals, showing whether difficulties are improving or persisting.

Overall, the dashboard may provide physicians with a comprehensive, data-driven view of patient progress, making it possible to identify successes, pinpoint areas of concern, and deliver more personalized and effective support.

### SOFTWARE IMPLEMENTATION

In further embodiments, the functionality of the BCST engine may be embodied as instructions stored on one or more non-transitory computer-readable media that, when executed by at least one processor, cause the processor to perform the operations disclosed herein. Such instructions may cause the processor to collect and analyze audio data to determine a history of cough events; to collect patient-reported cough data via a user interface; and to analyze both datasets to automatically generate and provide personalized recommendations for applying BCST. Additional instructions may enable detection of trends in cough events and patient-reported outcomes; identification of cough bouts and gaps with no coughing; generation of positive reinforcement messages and encouraging support messages; periodic progress reporting; identification of cough triggers and provision of patient-specific guidance; operation of visual, auditory, and haptic outputs; and implementation of machine learning, including reinforcement learning, to adaptively refine therapy recommendations based on ongoing user data. Instructions may further cause execution of a mobile application that communicates with a remote database and physician dashboard via an API.

### HARDWARE/SOFTWARE PARTITIONING

The BCST engine may be partitioned across on-device components and cloud services. For example, on-device modules may handle audio collection, feature extraction, and real-time prompting, while cloud modules may maintain longitudinal histories, model training, clinician dashboard services, and population-level optimization. In low-connectivity scenarios, essential recommendation logic may be cached on-device to ensure continuity of guidance, with periodic synchronization to the cloud when network connectivity is available.

### CLINICIAN OVERSIGHT

In embodiments involving a physician dashboard, authorized clinicians may remotely review patient progress, including objective cough metrics, PRO trends, and adherence metrics, and may optionally input clinician notes. Such clinician inputs may be recorded and auditable without departing from the automated nature of the recommendation engine.

### NON-TRANSITORY MEDIA

As used herein, a “non-transitory computer-readable medium” includes any tangible storage medium readable by a machine, including RAM, ROM, EEPROM, flash memory, optical disks, magnetic disks, or other magnetic storage devices, but excluding transitory propagating signals per se. Instructions stored on such media, when executed, cause the systems described herein to perform the disclosed methods.

### EXAMPLE OF USE

To demonstrate the efficacy of the digital therapeutic of the present invention, a proof-of-concept study was carried out using a prospective, decentralized design over a four-week intervention period. The study enrolled up to twenty English-speaking adults with refractory chronic cough (RCC), recruited remotely across both the United States and Europe. Participants were asked to continuously monitor cough activity for at least 20 hours per day, beginning with a baseline monitoring period of at least seven days, followed by a minimum of 28 days using the platform incorporating the present invention. During the intervention phase, participants engaged with the application's cough-management techniques, which included real-time feedback, tailored suppression coaching, and reinforcement strategies.

The study assessed both objective and qualitative endpoints. Objective measures included pre-and post-intervention cough frequency and cough bout rates, allowing for a clear comparison of symptom burden before and after engagement with the therapy. In addition, open-ended interviews provided insight into the patient experience, gathering feedback on usability, satisfaction, and perceived value of the program in daily life.

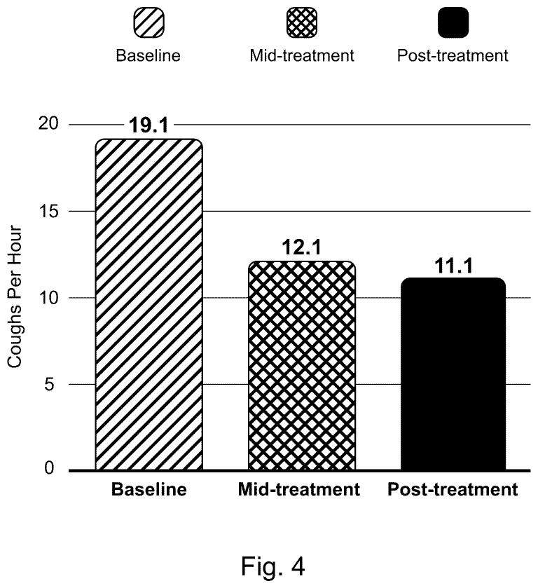

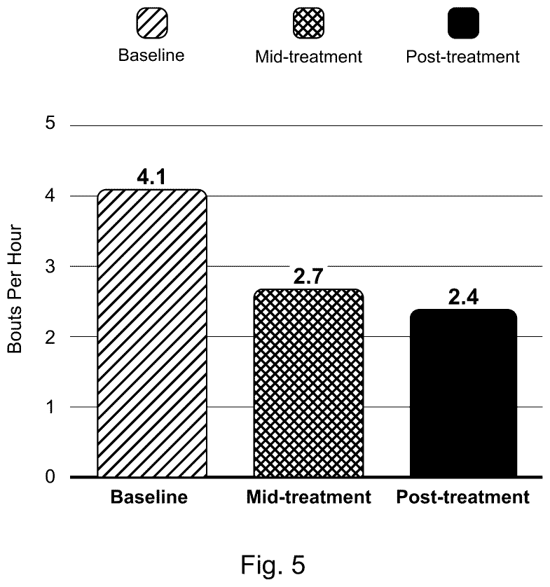

Preliminary findings have been highly encouraging. Results demonstrated a 41.8% reduction in cough frequency and a 41.5% reduction in cough bouts (see FIGS. 4 and 5), highlighting both the feasibility of a decentralized, app-based approach and the effectiveness of this digital therapeutic in reducing UCC/RCC symptoms. Importantly, these outcomes suggest that the method of the present invention not only replicates the benefits of traditional Behavioral Cough Suppression Therapy but may also enhance access, adherence, and scalability by leveraging technology for real-time monitoring and personalized guidance. These early results support the promise of the present invention as a viable, patient-centered solution for managing chronic cough.

The novel digital therapeutic method of the invention may provide the therapy in a new and interactive format, allowing patients to engage with the therapy at their convenience while still benefiting from evidence-based strategies and guidance. By integrating the Behavioral Cough Suppression Therapy into a digital medium, this novel approach has the potential to improve patient adherence, track progress, and provide real-time feedback, further enhancing its therapeutic efficacy.

### EXAMPLE OF USER INTERFACE ON A SMARTPHONE APPLICATION

As described above, the present disclosure relates to a digital cough suppression therapy system, which may be implemented as a software application operable on a smartphone, tablet, computer, or wearable device. The system may guide a user through a structured, multi-week behavioral therapy program designed to reduce cough frequency and improve voluntary control of the cough reflex. The program may be delivered through a sequence of interactive screens, modules, and exercises presented via a graphical user interface (GUI). Each stage of the therapy may correspond to a different week of the program, and may include educational materials, guided exercises, and progress-tracking tools.

The user interface may be configured to adapt to user responses, measured progress, and engagement level. The system may collect self-reported data, physiological input from sensors, or interaction metrics to personalize the therapy experience. In some implementations, the user interface may employ animations, icons, progress bars, and feedback cues to improve user comprehension and motivation. The following figures illustrate exemplary screenshots representing various stages of the six-week digital cough suppression therapy. While the illustrated example is 6 weeks in duration, the invention is not limited in this regard. A therapy session may be anywhere from 2 to 8 weeks long, depending on factors such as the doctor's recommendations, the health system context, payment arrangements (out-of-pocket, employer benefit, or reimbursement), adaptation based on results from early therapies, and other considerations.

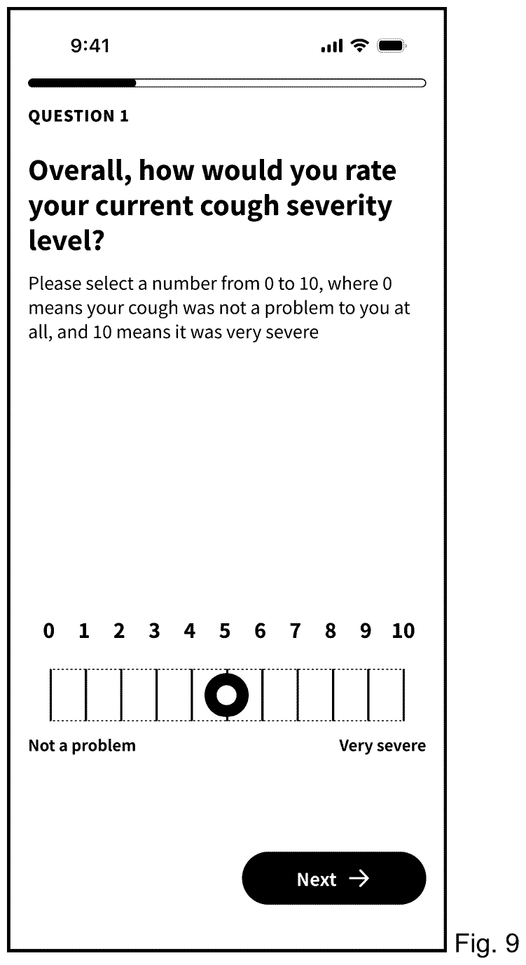

FIG. 9 is an exemplary screenshot showing an initial assessment stage of the digital cough suppression therapy. The interface may display assessment questions, baseline cough frequency tracking tools, and self-reported measures of cough impact or urge-to-cough intensity. The user may be prompted to input demographic data, medical history, and daily cough patterns to enable individualized therapy scheduling and goal calibration.

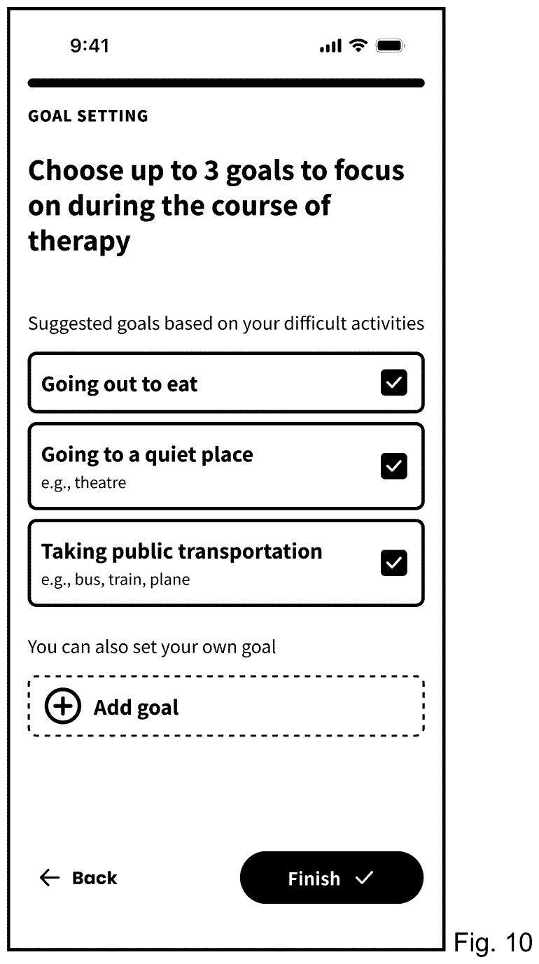

FIG. 10 is an exemplary screenshot showing a goal-setting stage of the therapy. The interface may present a guided workflow allowing the user to establish personal objectives for cough reduction, symptom management, and behavioral awareness. Visual indicators such as progress bars, sliders, or selectable milestones may allow the user to set achievable weekly goals, while background analytics may adapt subsequent content to the user's initial assessment.

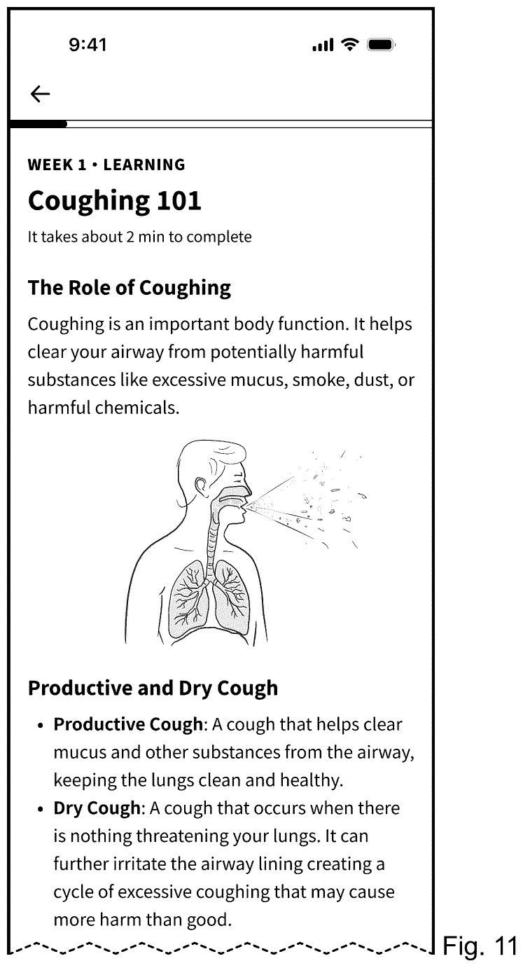

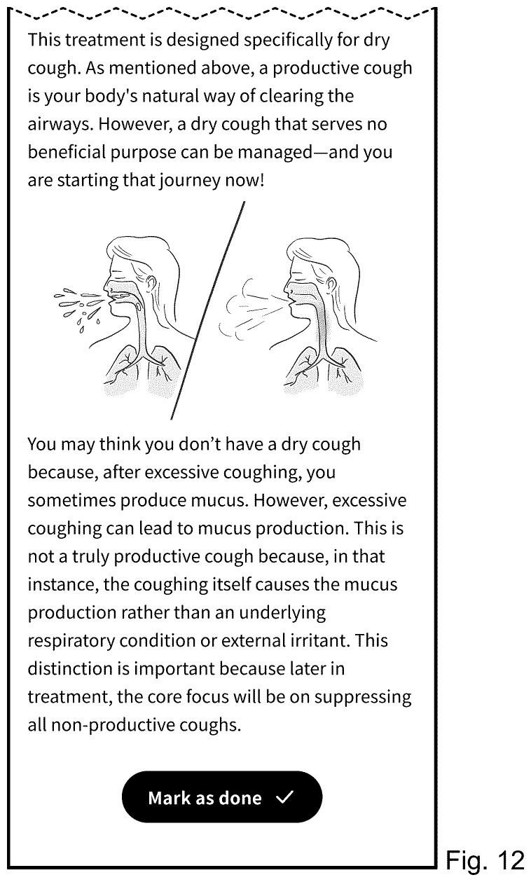

FIGS. 11 and 12 are exemplary panels showing a Week 1 learning, Cough 101, stage of the therapy. These panels may introduce educational content explaining the physiology of coughing and its habitual triggers. The panels may include interactive diagrams, narrated animations, or quiz-style modules enabling users to understand reflex pathways and cognitive influences on cough behavior.

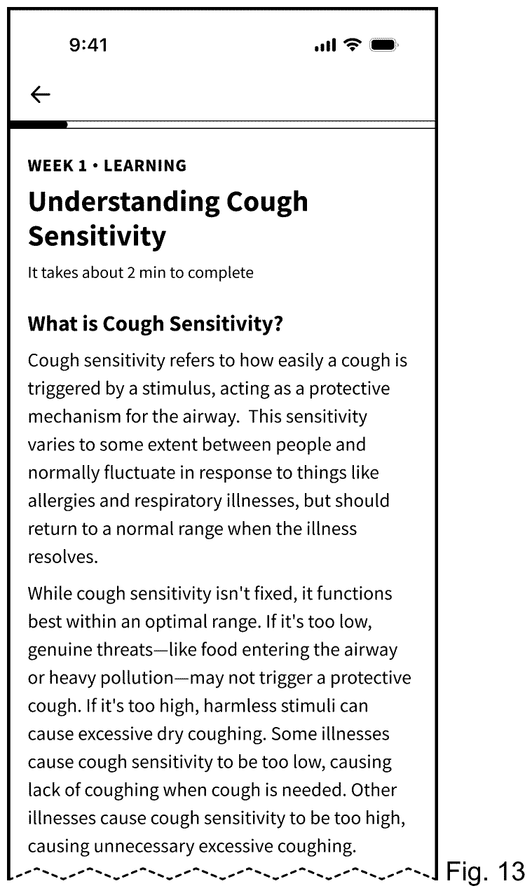

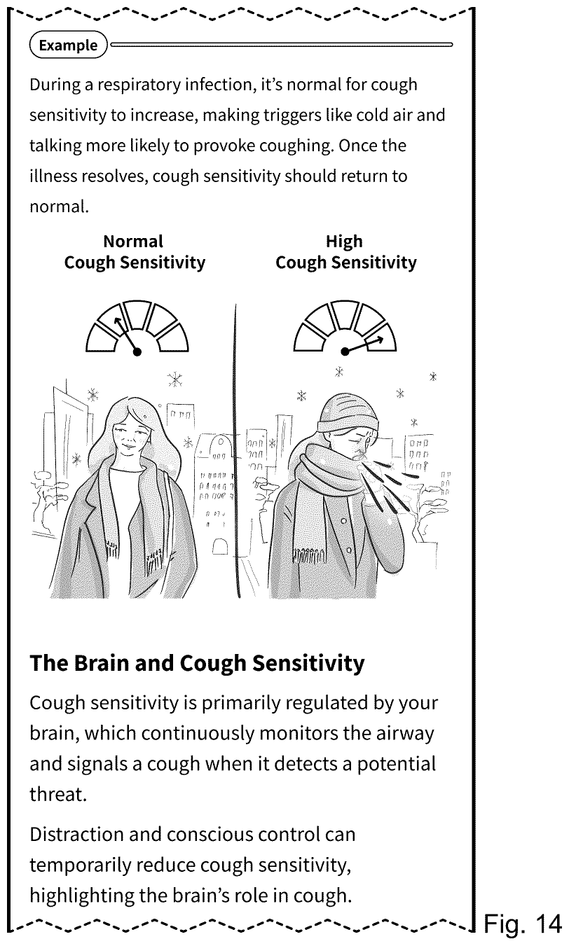

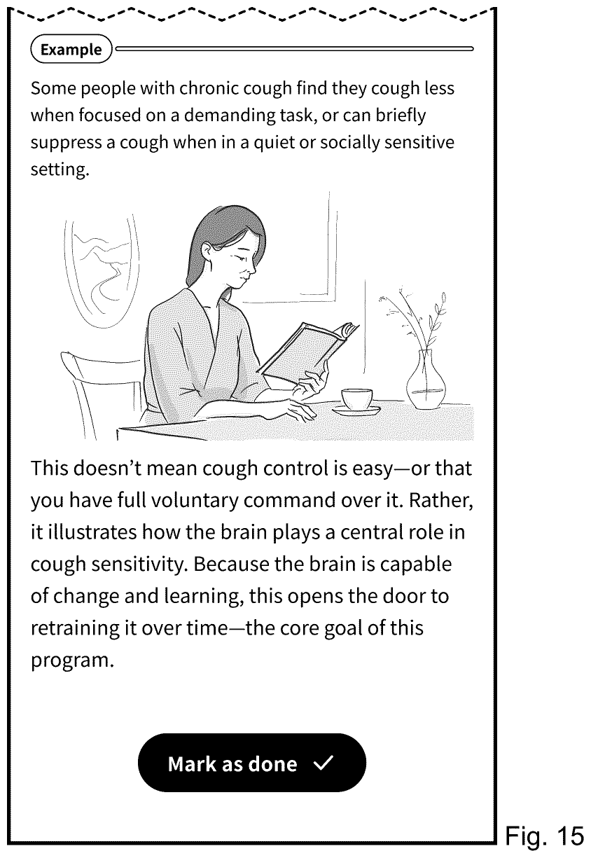

FIGS. 13 to 15 are exemplary panels showing a Week 1 learning, Understanding Cough Sensitivity, stage of the therapy. The interface may illustrate the relationship between cough sensitivity and environmental or psychological stimuli. The screens may include adjustable visualizations showing sensitivity thresholds, self-assessment sliders, or journaling prompts for users to reflect on their individual cough patterns and perceived triggers.

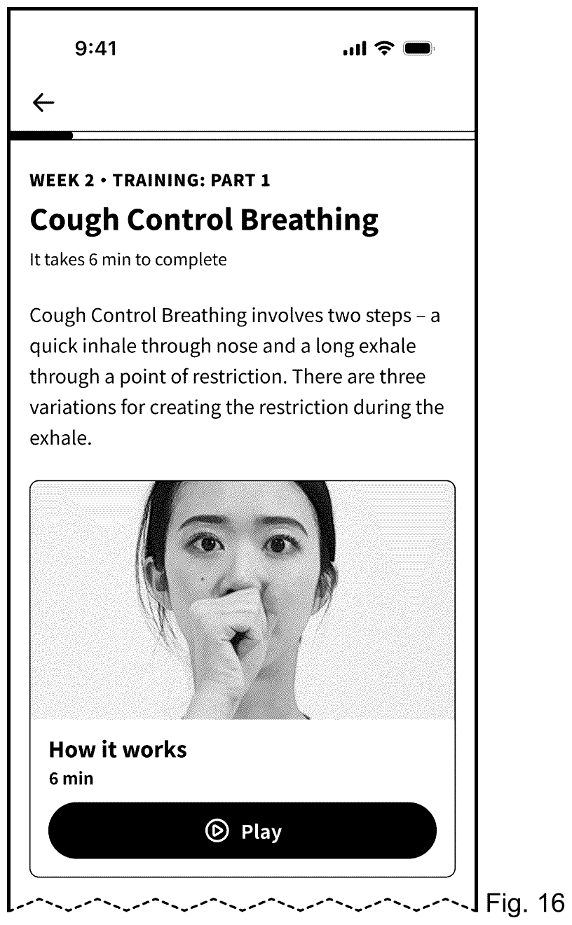

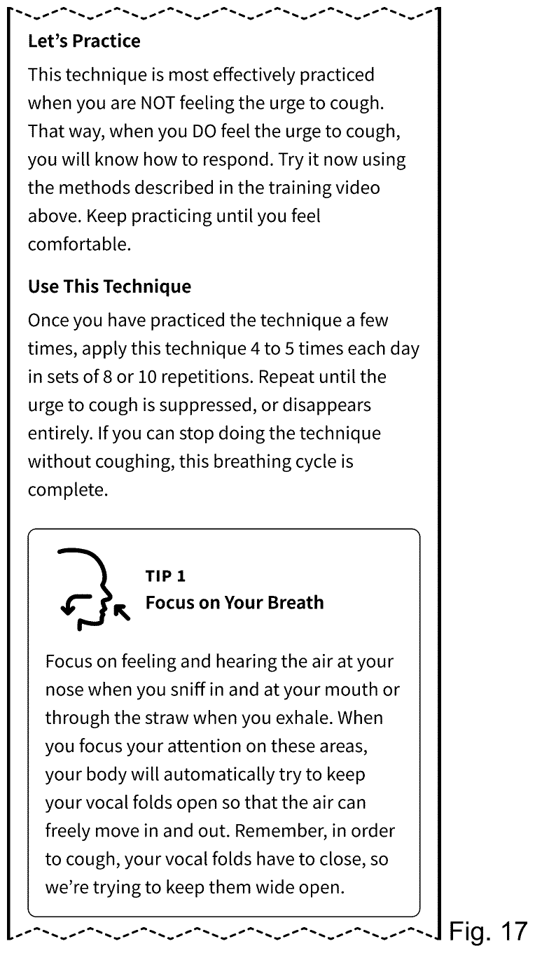

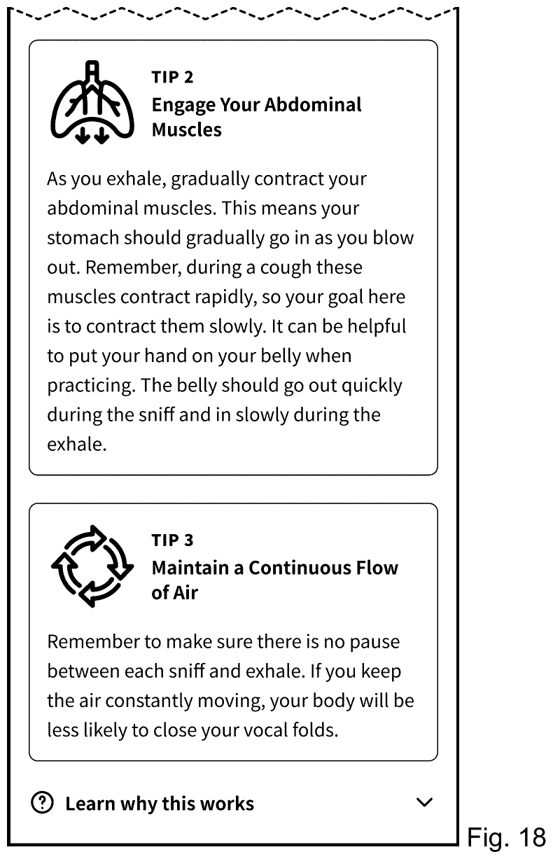

FIGS. 16 to 18 are exemplary panels showing a Week 2, Cough Control Breathing stage. The interface may display breathing exercises and timed inhalation/exhalation animations designed to interrupt the cough reflex. The system may provide real-time feedback through graphical cues, vibration, or audio pacing, and may allow the user to record perceived control over coughing urges.

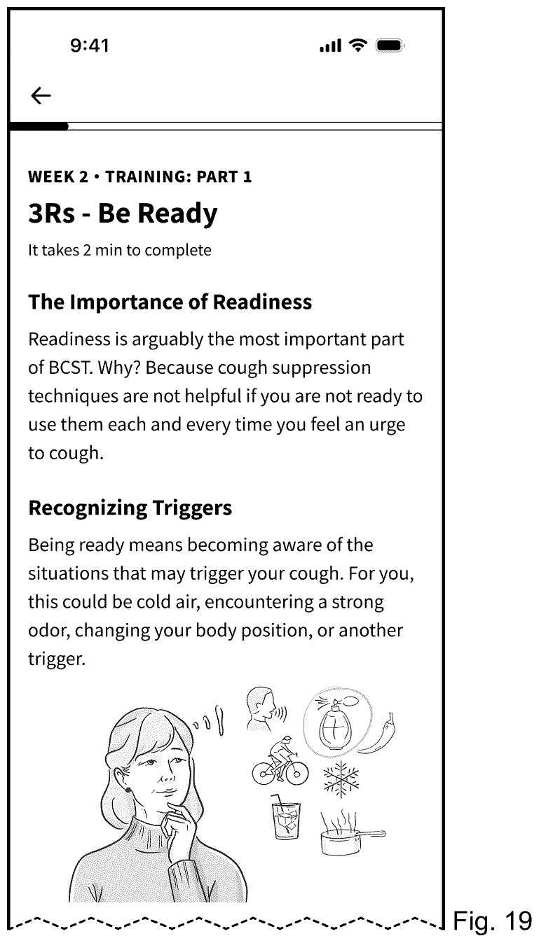

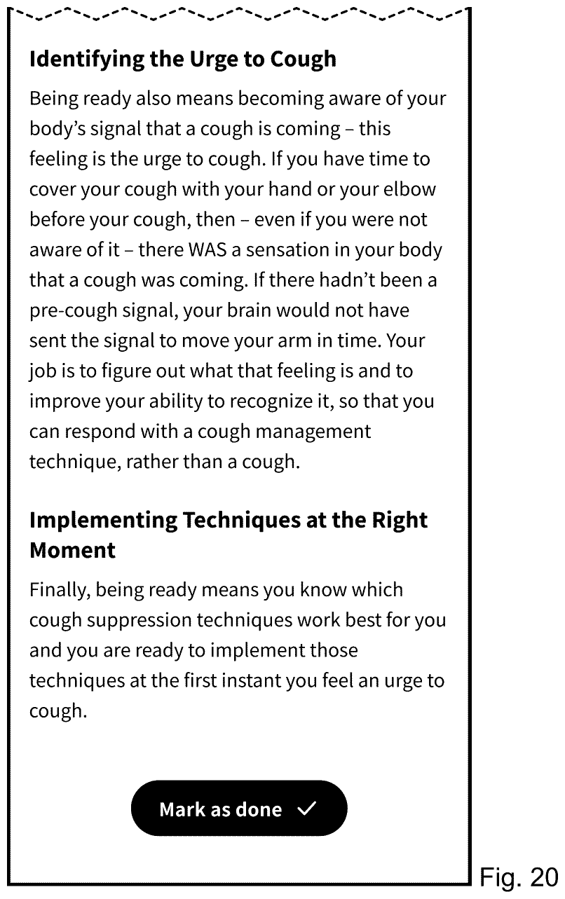

FIGS. 19 and 20 are exemplary panels showing a Week 2, Readiness to suppress when the urge to cough arises, stage of the therapy. The user interface may assess the user's preparedness to apply learned breathing techniques in real-world contexts. It may present readiness scales, motivational feedback, or a summary of progress achieved during the previous sessions.

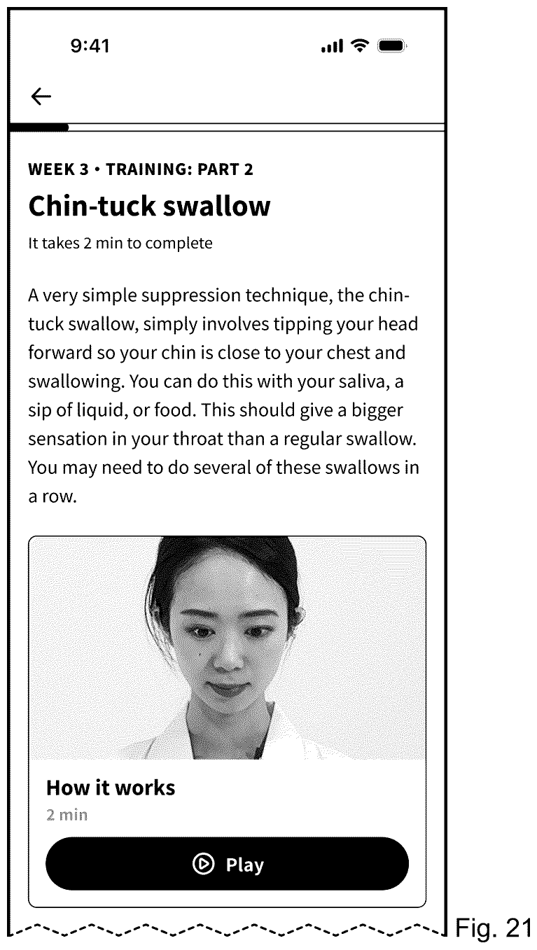

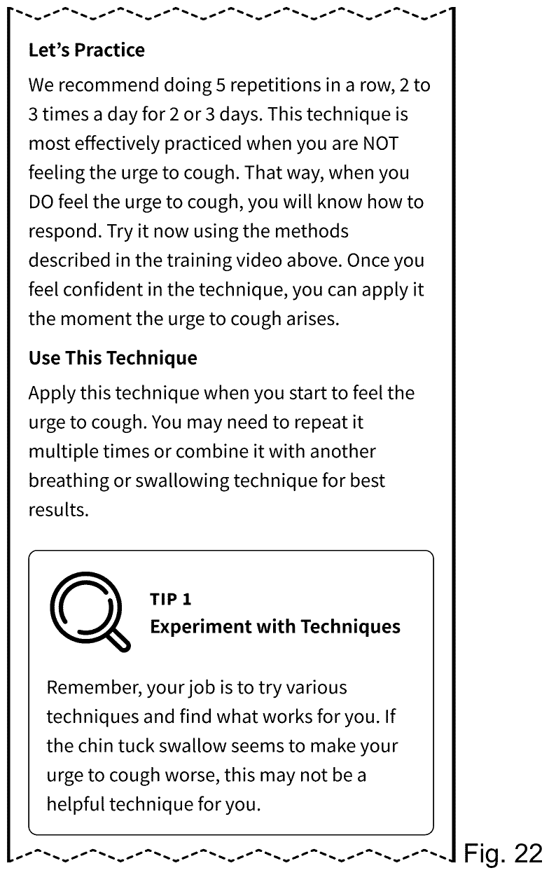

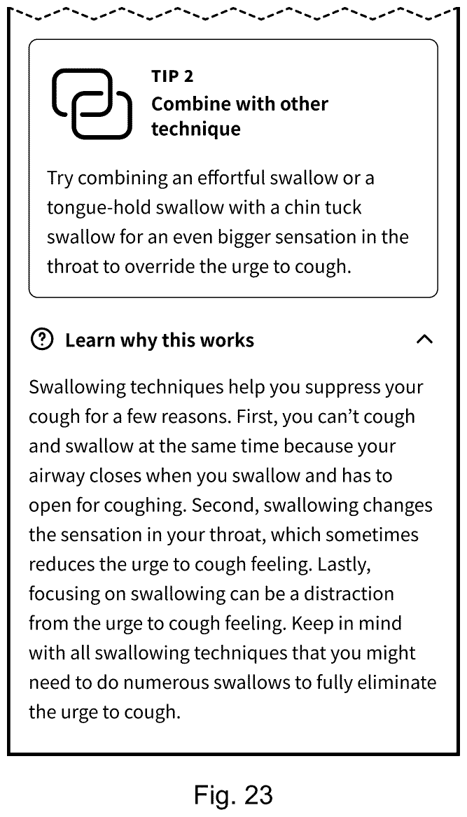

FIGS. 21 to 23 are exemplary panels showing a Week 3, Training: Part 1 stage of the therapy. These panels may guide the user through structured training sessions emphasizing controlled exposure to mild cough stimuli while applying breathing and suppression techniques. The interface may display dynamic progress indicators, reinforcement feedback, and optional pause or repeat functions.

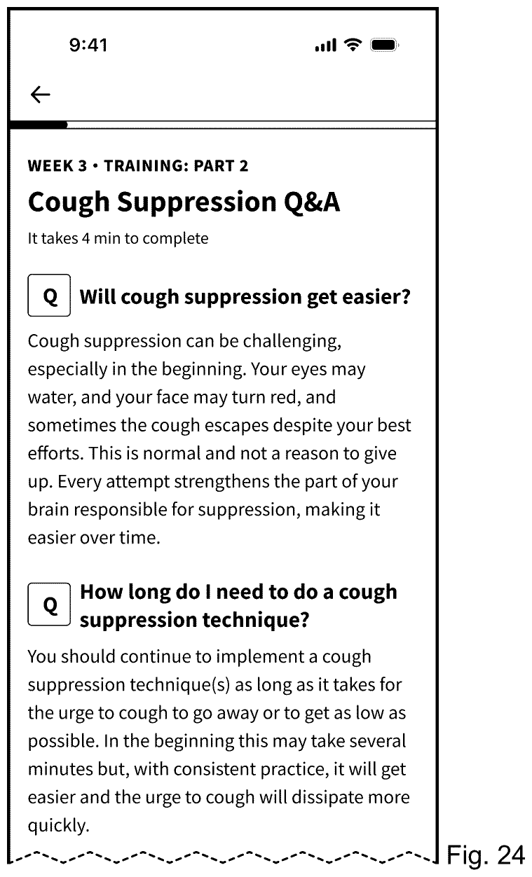

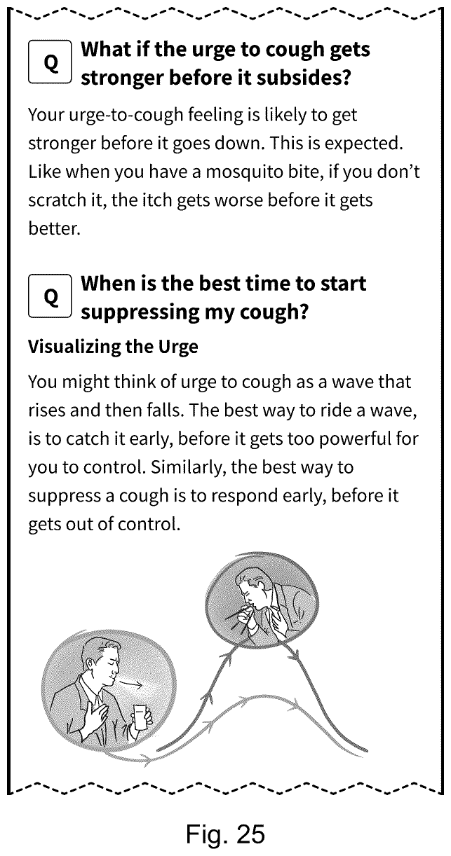

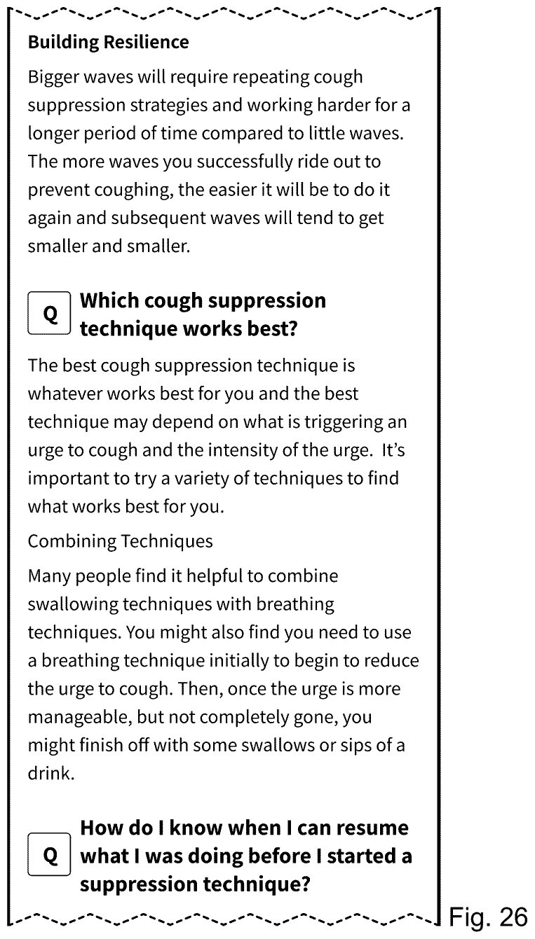

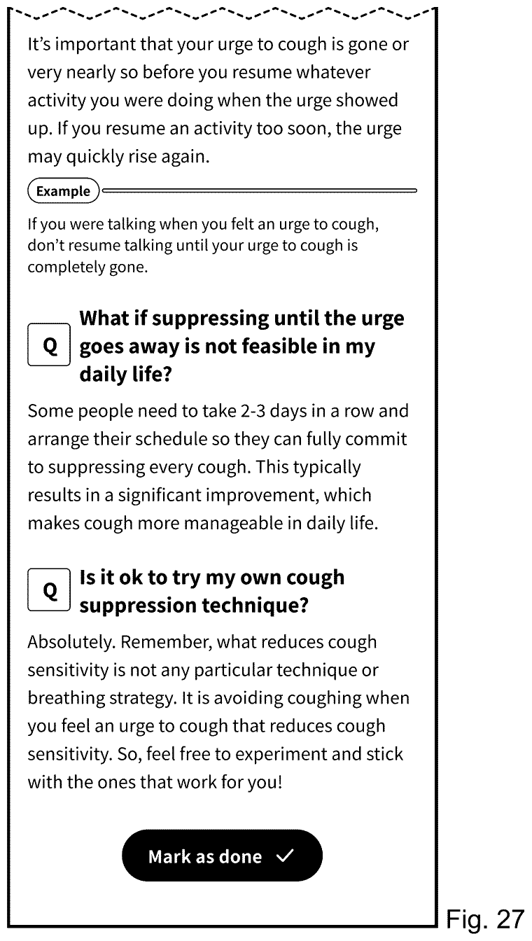

FIGS. 24 to 27 are exemplary panels showing a Week 3, Training: Part 2 stage of the therapy. The user interface may provide progressively challenging exercises or simulated triggers. The panels may include audiovisual cues representing coughing scenarios and may log the user's responses to refine personalized difficulty levels.

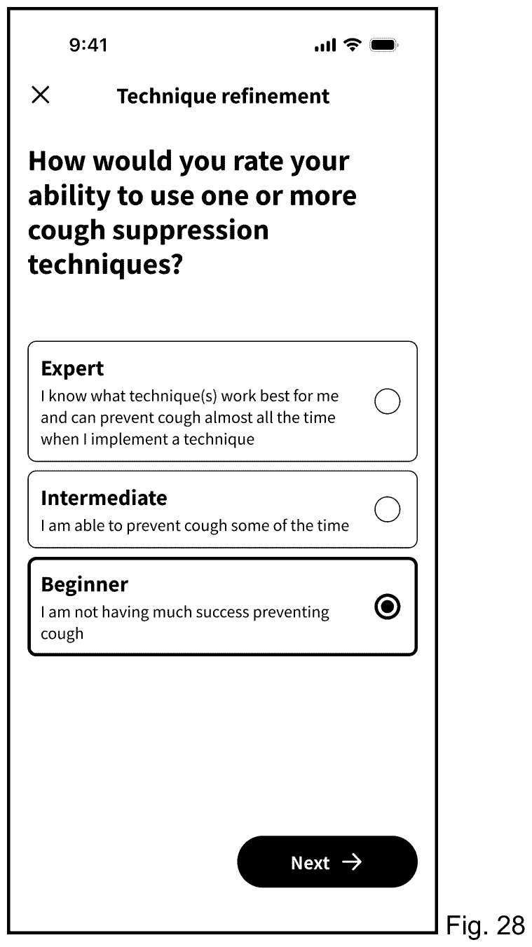

FIG. 28 is an exemplary screenshot showing a Week 4, Technique Refinement stage of the therapy. The interface may summarize user performance, identify patterns of successful cough suppression, and offer refined technique recommendations. Interactive graphs or achievement badges may be displayed to maintain user engagement.

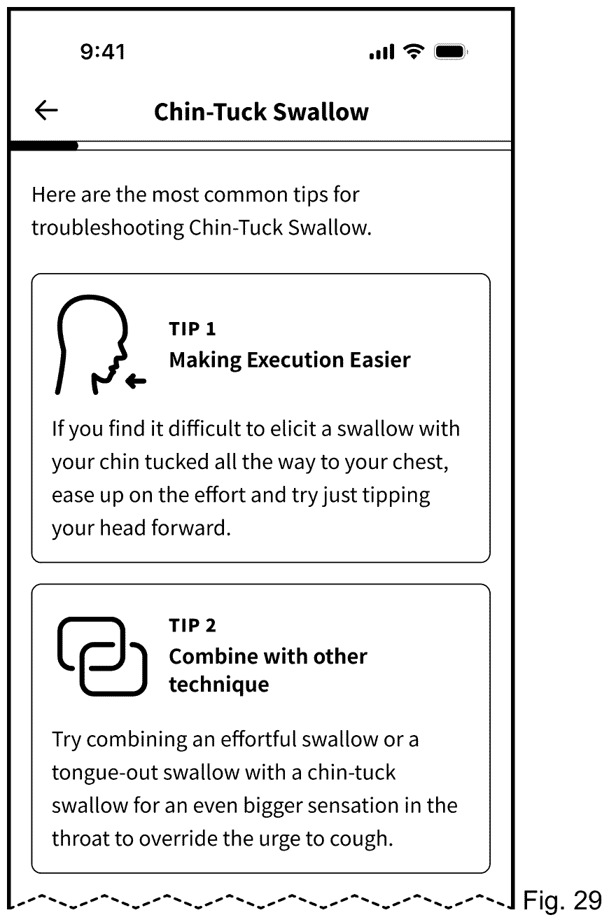

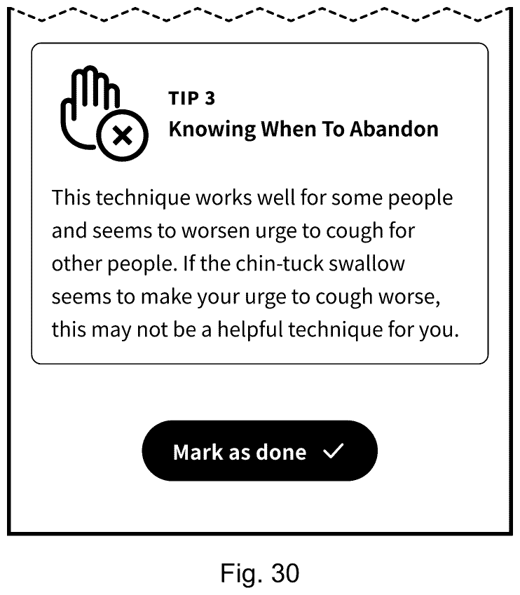

FIGS. 29 and 30 are exemplary panels showing a Week 4, Optimizing Progress stage of the therapy. The interface may display performance analytics comparing week-to-week improvement, offer reminders for consistency, and recommend individualized practice routines. The user may be encouraged to revisit earlier modules to strengthen weak areas.

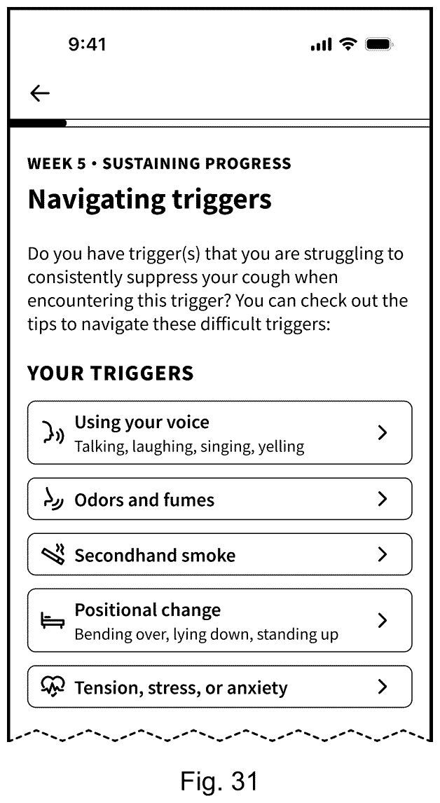

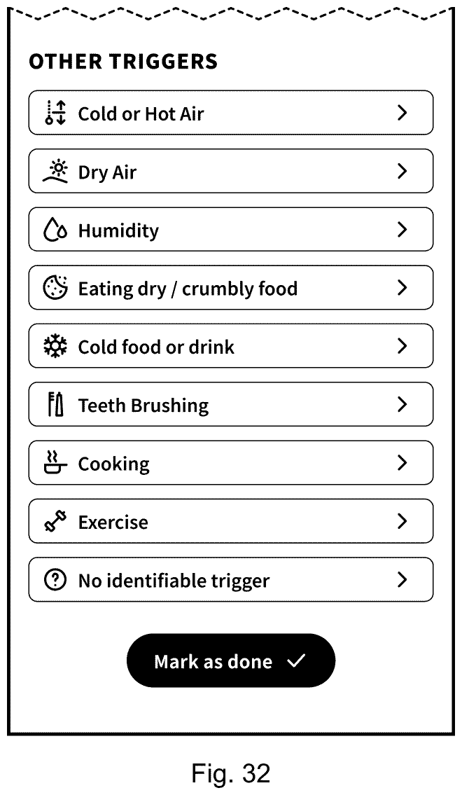

FIGS. 31 and 32 are exemplary panels showing a Week 5, Sustaining Progress: Navigating Triggers stage of the therapy. The screens may present coping strategies and cognitive-behavioral techniques to help the user identify and manage high-risk situations that typically provoke coughing. Scenario-based exercises and adaptive prompts may support long-term behavioral reinforcement.

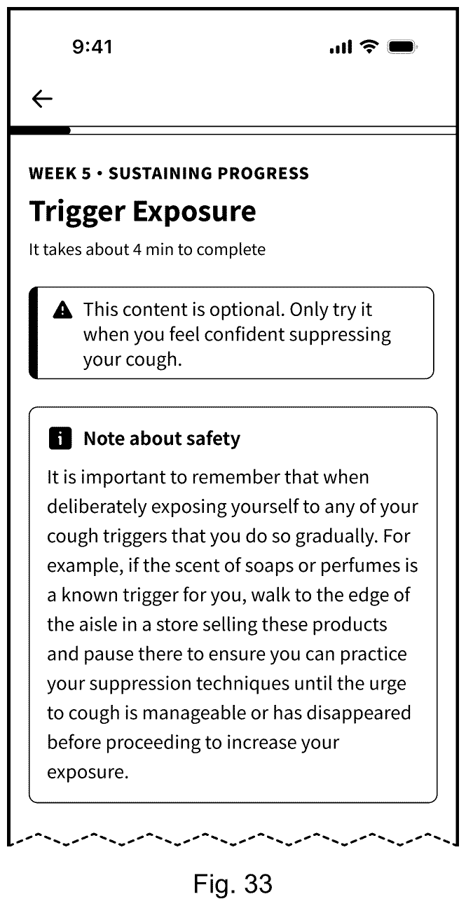

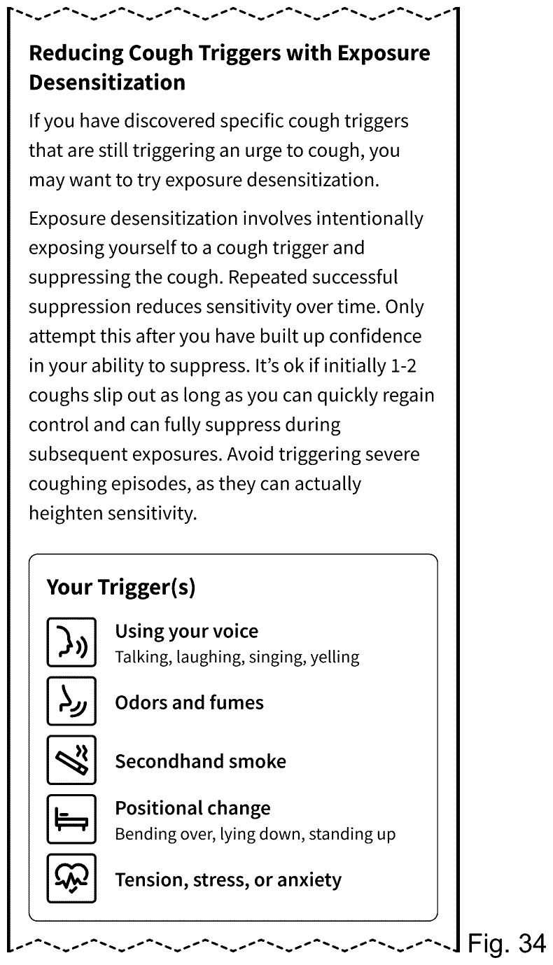

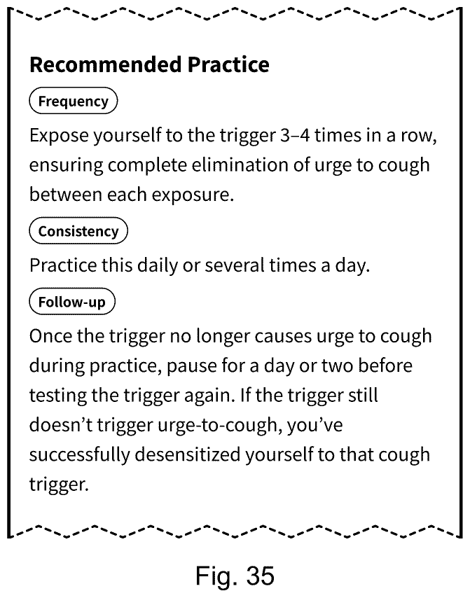

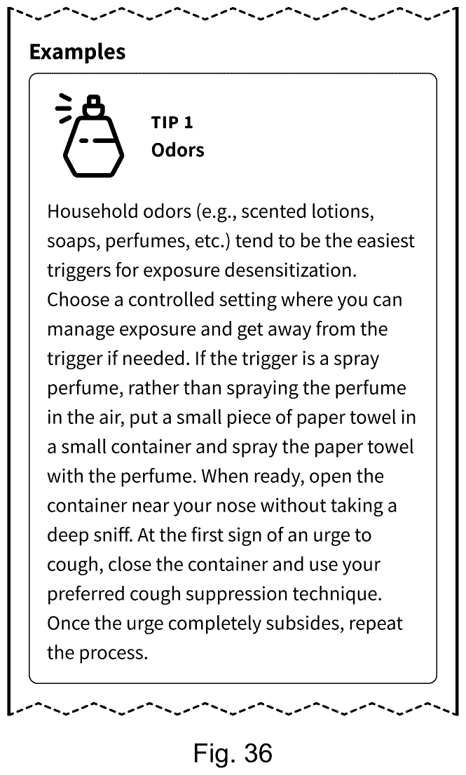

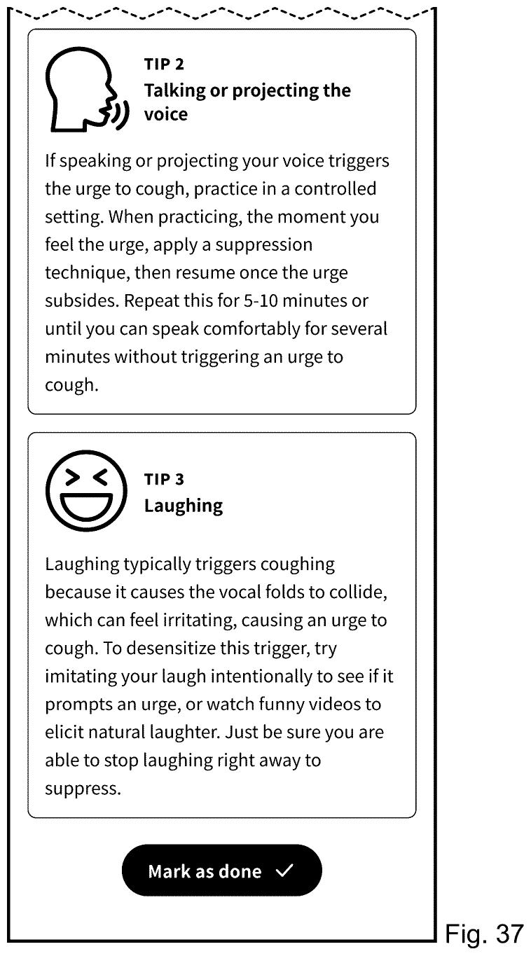

FIGS. 33 to 37 are exemplary panels showing a Week 5, Triggers Exposure stage of the therapy. The interface may simulate environmental or situational triggers through images, sounds, or descriptive prompts, encouraging the user to practice cough control under guided exposure. Progress indicators and self-report metrics may help evaluate the user's ability to maintain suppression during escalating challenges.

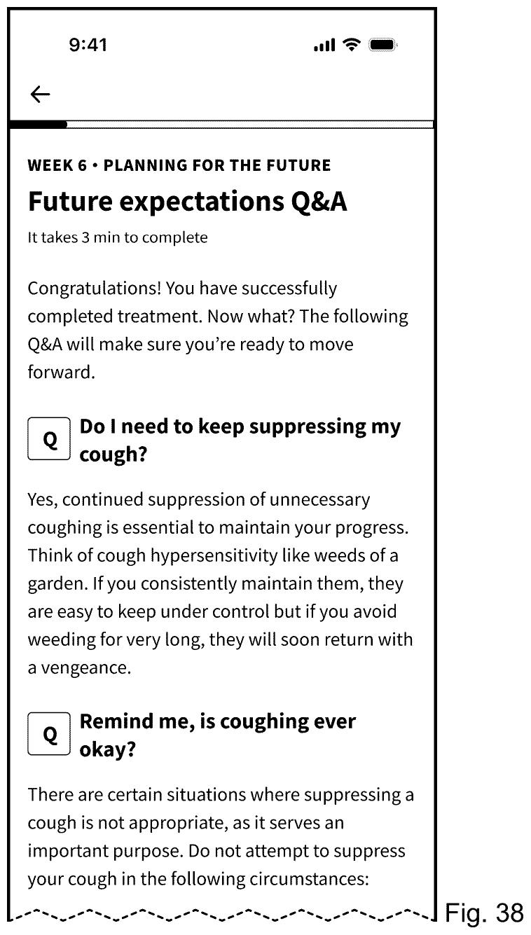

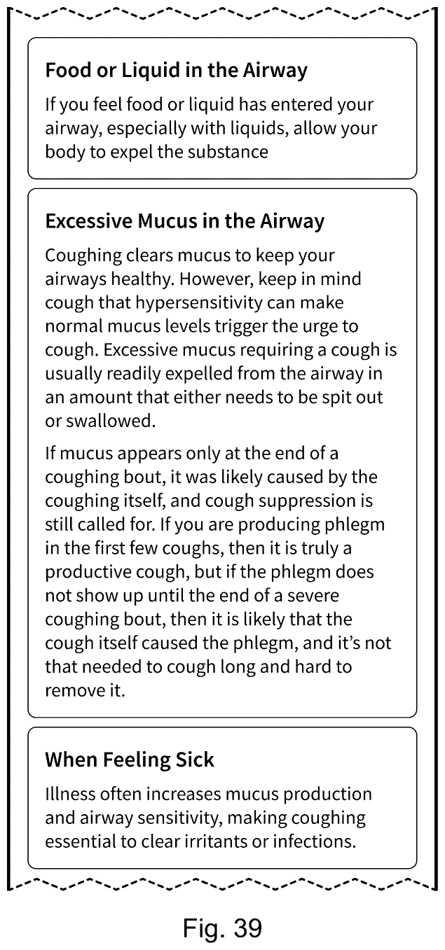

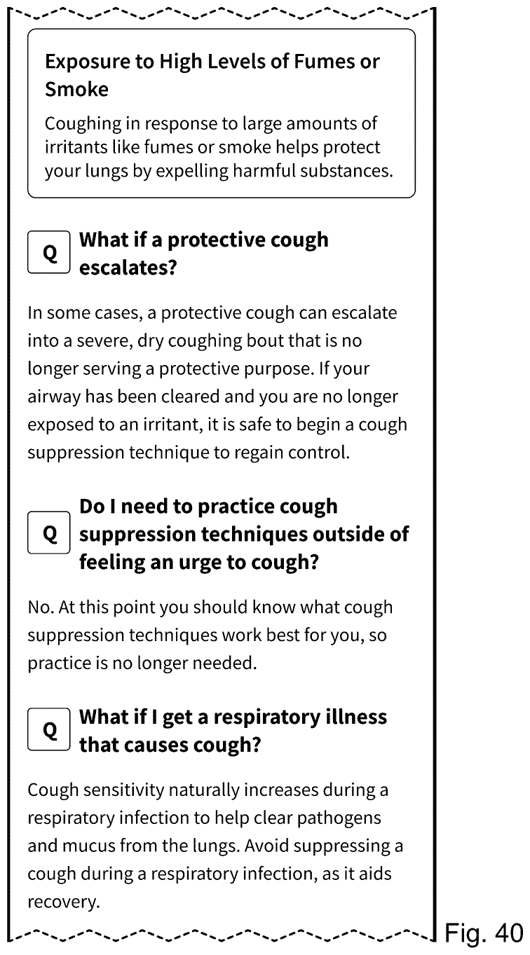

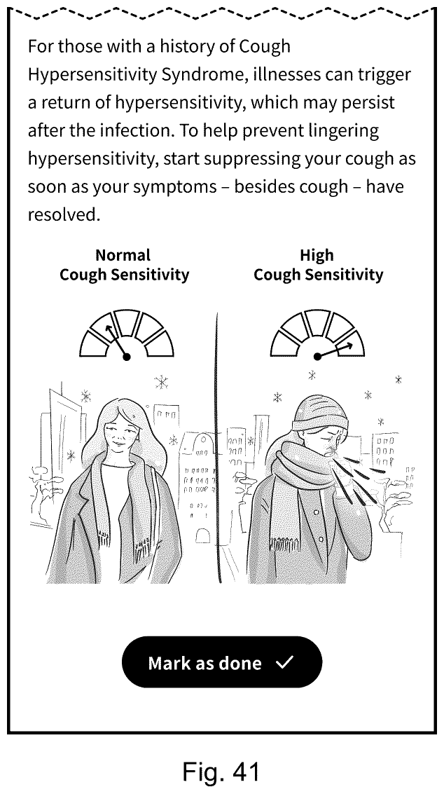

FIGS. 38 to 41 are exemplary panels showing a Week 6, Planning for the Future stage of the therapy. The interface may guide the user through relapse-prevention planning, long-term progress tracking, and the creation of personalized maintenance strategies. It may include summary dashboards, motivational messages, and options for exporting or sharing progress reports with healthcare providers.

For the purposes of this description, the term “personalized recommendations” is used to describe any one or a combination of messages describing therapy guidance, BCST therapy instructions, and support/motivational messages. The term “objective cough tracking data” is used to describe objectively measured and recorded cough data, such as audio captured through a microphone on a smartphone or other electronic device. This includes data on automatically detected cough events using continuous audio monitoring and signal analysis. Analysis may also include more detailed information such as cough trends, coughing episodes, and cough-free intervals. “Cough events” refers to acoustic events that are identified by exceeding a sound energy threshold, confirmed through frequency-time domain analysis, and distinguished from non-cough sounds (e.g., using neural networks). “Patient-specific guidance” describes specific advice or instruction tailored to the individual patient on how to manage or avoid their identified cough triggers.

It is contemplated that any embodiment discussed in this specification can be implemented with respect to any method of the invention, and vice versa. It will be also understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. Incorporation by reference is limited such that no subject matter is incorporated that is contrary to the explicit disclosure herein, no claims included in the documents are incorporated by reference herein, and any definitions provided in the documents are not incorporated by reference herein unless expressly included herein.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps, as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12, 15, 20 or 25%.

All of the devices and/or methods disclosed and claimed herein can be made and executed without undue experimentation in light of the present disclosure. While the devices and methods of this invention have been described in terms of preferred embodiments, it will be apparent to those of skill in the art that variations may be applied to the devices and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A method of automatically generating and providing personalized recommendations for applying Behavioral Cough Suppression Therapy, the method comprising the following steps:
a. collecting objective cough tracking data by operating an electronic device having a microphone and (i) configured to communicate sound detected by the microphone to an external processor or (ii) comprising an internal processor configured to analyze sound detected by the microphone and determine a history of cough events therefrom,
b. collecting subjective patient-reported cough data by providing a user interface configured for entry of subjective patient-reported cough data, and
c. operating the external processor or the internal processor to analyze both objective cough tracking data and subjective patient-reported cough data to automatically generate and provide personalized recommendations for applying Behavioral Cough Suppression Therapy.

2. The method of providing personalized Behavioral Cough Suppression Therapy, as in claim 1, wherein the electronic device is a smart ring, a smartwatch, or a smartphone.

3. The method of providing personalized Behavioral Cough Suppression Therapy, as in claim 1, wherein the user interface configured for entry of subjective patient-reported cough data in step (b) is further configured to collect the subjective patient-reported cough data via at least one input of the electronic device of step (a).

4. The method of providing personalized Behavioral Cough Suppression Therapy, as in claim 3, wherein the processor of the electronic device is further configured to automatically conduct step (c) of analyzing objective cough tracking data and subjective patient-reported cough data to automatically generate and provide personalized recommendations for applying Behavioral Cough Suppression Therapy.

5. The method of providing personalized Behavioral Cough Suppression Therapy, as in claim 1, further comprising a step (d) of detecting a trend in cough events and adjusting the recommendations for applying the Behavioral Cough Suppression Therapy based thereon.

6. The method of providing personalized Behavioral Cough Suppression Therapy, as in claim 1, wherein step (d) further comprises a step of detecting a trend in cough events, detecting a trend in subjective patient-reported cough outcomes, recording a history of following Behavioral Cough Suppression Therapy recommendations, and adjusting the recommendations for applying Behavioral Cough Suppression Therapy based thereon.

7. The method of providing personalized Behavioral Cough Suppression Therapy, as in claim 1, wherein step (c) further comprises a step of analyzing objective cough tracking data to detect at least one of: a cough trend, a cough bout, and a gap with no coughing.

8. The method of providing personalized Behavioral Cough Suppression Therapy, as in claim 1, wherein the electronic device comprises a display or a speaker, and the processor of the electronic device is configured to present personalized recommendations for applying Behavioral Cough Suppression Therapy using the display and/or the speaker of the electronic device.

9. The method of providing personalized Behavioral Cough Suppression Therapy, as in claim 1, wherein step (b) of collecting subjective patient-reported cough data includes electronically collecting subjective patient-reported Outcomes.

10. The method of providing personalized Behavioral Cough Suppression Therapy, as in claim 5, further comprises a step (e) of detecting patient adherence to recommended Behavioral Cough Suppression Therapy via providing the user interface configured for entering and recording a history of performing recommended BCST cough suppression training sessions.

11. The method of providing personalized Behavioral Cough Suppression Therapy, as in claim 10, wherein at least one cough suppression training session is guided by the electronic device.

12. The method of providing personalized Behavioral Cough Suppression Therapy, as in claim 5, wherein step (d) further comprises generating encouraging messages upon detecting a subsiding trend of cough events.

13. The method of providing personalized Behavioral Cough Suppression Therapy, as in claim 11, wherein step (d) further comprises generating encouraging support messages upon detecting an increasing trend of cough events while maintaining adherence to recommended Behavioral Cough Suppression Therapy.

14. The method of providing personalized Behavioral Cough Suppression Therapy, as in claim 11, wherein step (d) further comprises a step of generating a progress report on a periodic basis.

15. The method of providing personalized Behavioral Cough Suppression Therapy, as in claim 11, wherein step (d) further comprises a step of identifying and reporting at least one cough trigger.

16. The method of providing personalized Behavioral Cough Suppression Therapy, as in claim 15, wherein step (d) further comprises a step of providing patient-specific guidance on how to navigate the at least one cough trigger.

17. The method of providing personalized Behavioral Cough Suppression Therapy, as in claim 1, wherein step (c) of developing personalized recommendations for applying Behavioral Cough Suppression Therapy comprises a step of using a Behavioral Cough Suppression Therapy engine.

18. The method of providing personalized Behavioral Cough Suppression Therapy, as in claim 17, wherein in step (c) the Behavioral Cough Suppression Therapy engine is trained and operated using a machine-learning technique.

19. The method of providing personalized Behavioral Cough Suppression Therapy, as in claim 1, wherein the electronic device is further equipped with a vibration generator, and wherein step (c) further comprises a step of generating haptic signals or audio signals by the electronic device as a reminder to practice recommended cough suppression techniques.

20. The method of providing personalized Behavioral Cough Suppression Therapy, as in claim 1, wherein step (a) is automatically conducted for at least 10 hours per day.

21. The method of providing personalized Behavioral Cough Suppression Therapy, as in claim 1, wherein step (a) is preceded by a step of collecting baseline cough data when no cough suppression therapy is applied.

22. The method of providing personalized Behavioral Cough Suppression Therapy, as in claim 21, wherein the step of collecting baseline cough data is conducted automatically for at least 3 days prior to initiation of Behavioral Cough Suppression Therapy.

23. A system for automatically generating and providing personalized recommendations for applying Behavioral Cough Suppression Therapy, the system comprising:
an electronic device having a microphone configured to detect ambient sound and generate audio data corresponding to cough events, at least one processor configured to analyze the audio data to determine a history of cough events, a user interface configured to collect subjective patient-reported cough data, and a Behavioral Cough Suppression Therapy engine configured to analyze both the history of cough events and the subjective patient-reported cough data to automatically generate and provide personalized recommendations for applying Behavioral Cough Suppression Therapy.

24. A non-transitory computer-readable medium storing instructions that, when executed by at least one processor, cause the processor to perform a method of automatically generating and providing personalized recommendations for applying Behavioral Cough Suppression Therapy, the method comprising:
collecting objective cough tracking data by operating an electronic device having a microphone configured to detect ambient sound and generate audio data corresponding to cough events,
analyzing the audio data to determine a history of cough events,
collecting subjective patient-reported cough data via a user interface, and
analyzing both the history of cough events and the subjective patient-reported cough data to automatically generate and provide personalized recommendations for applying Behavioral Cough Suppression Therapy.
